(19) United States
(12) Patent Application Publication
(10) Pub. No.: US 2008/0221116 A1
(43) Pub. Date: Sep. 11, 2008

(54) ISOXAZOLINE ALPHA 1a/1d ADRENORECEPTOR ANTAGONISTS

(76) Inventors: Ellen W. Baxter, Glenside, PA (US); Samuel O. Nortey, Elkins Park, PA (US); Allen B. Reitz, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/855,757

(22) Filed: Sep. 14, 2007

Related U.S. Application Data
(60) Provisional application No. 60/825,730, filed on Sep. 15, 2006.

(51) Int. Cl. A61K 31/496; C07D 413/14

(52) U.S. Cl. 514/253.1; 544/367; 514/254.04; 544/364

(57) ABSTRACT

The present invention relates to isoxazolines useful as selective α₁ₐ/α_{1d} adrenoreceptor antagonists for the treatment of benign prostatic hypertrophy and/or lower urinary tract symptoms as well as to pharmaceutical compositions comprising said compounds, processes to prepare these compounds, and the use of these compounds as α₁ₐ/α_{1d} adrenoreceptor modulators in a method of treatment.

### CROSS-REFERENCE TO RELATED APPLICATIONS

This present application claims benefit of U.S. Provisional Patent Application Ser. No. 60/825,730, filed Sep. 15, 2006, which is incorporated herein by reference in its entirety and for all purposes.

### FIELD OF THE INVENTION

The present invention relates to new compounds, more particularly new isoxazolines as selective α₁ₐ/α_{1d} adrenoreceptor antagonists for the treatment of benign prostatic hypertrophy and/or lower urinary tract symptoms. The present invention also relates to pharmaceutical compositions comprising said new compounds, new processes to prepare these new compounds, to the use of these compounds as α₁ₐ/α_{1d} adrenoreceptor modulators and new uses as a medicine as well as method of treatments.

### BACKGROUND OF THE INVENTION

The adrenergic receptors (ARs), through which norepinephrine and epinephrine exert their biological activities, are targets for many therapeutically important drugs. The α1-ARs play a dominant role in control of smooth muscle contraction and are important in control of blood pressure, nasal congestion, prostate function, and other processes. Three genes encoding different α1-AR subtypes (α₁ₐ, α_{1b}, and α_{1d}) have been cloned for a number of species. These three cloned α1-ARs are best differentiated from one another on the basis of the relative binding affinities of a series of antagonist compounds. There is general agreement that the α₁ₐ- and α_{1b}-ARs correspond to the pharmacologically defined α₁ₐ- and α_{1b}-ARs, while the functional role of the α_{1d}-AR is less clear, although it appears to mediate contraction of certain blood vessels.

Benign prostatic hyperplasia (BPH) is a non-malignant enlargement of the prostate and is the cause of lower urinary tract symptoms (LUTS) in a large segment of the male population. Symptoms such as straining, hesitancy, dribbling, weak stream, and incomplete emptying are classified as voiding or obstructive symptoms. Obstructive symptoms are primarily due to pressure upon the urethra from the physical mass of the enlarged prostate gland (the static component) and the increased tone of the smooth muscle of the prostate stroma and bladder neck (the dynamic component). Irritative or storage symptoms associated with BPH are frequency, urgency, nocturia, dysuria, and burning sensation. Patients feel that these symptoms are more disturbing than the obstructive symptoms. As the urine flow is reduced, due to the bladder outlet obstruction, the wall around the bladder base thickens and becomes hyperactive.

Originally introduced as antihypertensive agents, α1-AR antagonists have become increasingly important in the management of BPH. α1-AR antagonists reduce smooth muscle tone in the prostate and lower urinary tract, thereby relaxing the bladder outlet and increasing urinary flow. The major disadvantage of non-selective α1-blockers is their adverse effect profile, particularly vasodilatation leading to dizziness, postural hypotension, asthenia, and occasionally syncope. For this reason, it would be desirable to block α1-ARs in the lower urinary tract without antagonizing the α1-ARs responsible for maintaining vascular tone.

A number of factors can be involved in lower urinary tract symptoms. Adrenergic stimulation of the bladder results in relaxation due to β-ARs, which dominate over contraction-mediating α1-ARs. Bladder contraction is primarily mediated by muscarinic receptors.

Spinal α1-ARs may be important targets for pharmacological treatment of BPH symptoms in humans. All three α1-AR subtype mRNAs are found throughout the human spinal cord, however the α_{1d} subtype mRNA is present at twice the level of the other subtypes, particularly in the ventral sacral motor neurons and autonomic parasympathetic pathways. There may be clinical advantages to the pharmacological blockade of the α_{1d}-ARs in the CNS in reducing BPH symptoms.

Antagonism of α_{1d}-ARs in the CNS and bladder may be an important activity in reducing the irritative or filling symptoms of BPH and improving patient symptom scores.

A uroselective, cardiovascular-sparing α1-AR antagonist would be expected to provide symptomatic relief of BPH comparable to currently marketed non-selective agents such as terazosin/Hytrin®, doxazosin/Cardura®, alfuzosin/Xatral®/Uroxatral® and weakly selective tamsulosin/Flomax®/Hamal®, without the undesirable side effects of postural hypotension, dizziness, and syncope. Ejaculatory dysfunction, or retrograde ejaculation, is a side effect seen in a number of patients using tamsulosin. This activity has been attributed to tamsulosin antagonism at the 5-HT_{1A} receptor. This often leads to discontinuation of treatment. Furthermore, the non-selective α1-AR antagonists and tamsulosin are contraindicated for use in conjunction with PDE inhibitors. There is likely to be high co-morbidity between LUTS and erectile dysfunction patients. Patients being treated for LUTS with the current α1-AR blockers will find that they are excluded from using PDE inhibitors. An α1-AR antagonist with a receptor subtype binding profile, which is selective for the α₁ₐ and α_{1d}, subtypes, but with relatively little antagonism of the α_{1b} subtype may effectively treat both obstructive and irritative symptoms of BPH. Such a compound is likely to have a low cardiovascular side effect profile and allow for use in conjunction with PDE inhibitors. Also low binding activity at the 5-HT_{1A} receptor is likely to reduce the incidence of ejaculatory side effects.

LUTS also develop in women of a certain age. As in men, LUTS in women include both filling symptoms such as urgency, incontinence and nocturnia, and voiding symptoms such as weak stream, hesitancy, incomplete bladder emptying and abdominal straining. The presence of this condition both in men and women suggests that at least part of the aetiology may be similar in the two sexes.

Accordingly, there is a need to provide dual selective α₁ₐ/α_{1d} adrenoreceptor antagonists, in other words compounds that interact both with the α₁ₐ and α_{1d} receptor but do not interact (or at least interact substantially less) with the α_{1b} receptor. The compounds of this invention are believed to be more efficacious drugs mainly for BPH/LUTS patients, and at the same time these compounds should show less unwanted side effects than the existing pharmaceuticals.

J. Med. Chem. 1995, 38, 4198-4210 discloses 4,5-dihydro-5-[[4-[2-(1-methylethoxy)phenyl]-1-piperazinyl]methyl]-3-isoxazolemethanol (compound 49 in the article) and 1-[[4,5-dihydro-5-[[4-[2-(1-methylethoxy)phenyl]-1-piperazinyl]methyl]-3-isoxazolyl]methyl]-piperidin-2-one perchlorate hydrate (compound 50 in the article and Compound 1, as a free base, herein). 4,5-Dihydro-5-[[4-[2-(1-methylethoxy)phenyl]-1-piperazinyl]methyl]-3-isoxazolemethanol is described as having strong Conditioned Avoidance Response (CAR) activity and a lack of D₂ binding and 5-HT_{1A}. 1-[[4,5-Dihydro-5-[[4-[2-(1-methylethoxy)phenyl]-1-piperazinyl]methyl]-3-isoxazolyl]methyl]-piperidin-2-one perchlorate hydrate on the other hand is described as being not so active in CAR but having a moderately high binding affinity for the 5-HT_{1A} site.

PCT patent application WO 93/04682 describes related benzamidomethyl aryl piperidines and piperazines as antipsychotic agents.

### SUMMARY OF THE INVENTION

A first aspect of the present invention are novel compounds of Formula (I) or a form thereof:

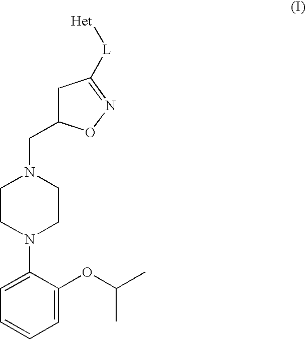

wherein
L is selected from the group consisting of —CH₂—, —CO—, and —CONH—;
Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with C₁₋₆alkyl, amino di-substituted with C₁₋₆alkyl, amino mono-substituted with ArC₁₋₆alkyl, amino disubstituted with ArC₁₋₆alkyl, Ar, Ar—CO, Ar-oxy, and Ar—SO₂;
Ar is selected from pyridinyl, phenyl and phenyl substituted with one or more substituents selected from halo, C₁₋₆alkyl, and C₁₋₆alkyloxy;
with the proviso that 1-[[4,5-dihydro-5-[[4-[2-(1-methylethoxy)phenyl]-1-piperazinyl]methyl]-3-isoxazolyl]methyl]-2-piperidone Compound 1 is not included.

A second aspect of this invention is a composition or medicament comprising one or more compounds of Formula (I) or a form thereof.

A third aspect of this invention is a method of synthesizing compounds of Formula (I) or a form thereof.

A fourth aspect of this invention is the use of one or more compounds of Formula (I) or a form thereof as selective α₁ₐ/α_{1d} adrenoreceptor antagonists.

A fifth aspect of this invention is a method for ameliorating, treating or preventing treatment of benign prostatic hypertrophy and/or lower urinary tract symptoms.

A sixth aspect of this invention is a method for use of one or more compounds of Formula (I) or a form thereof in the preparation of a composition or medicament for preventing, treating or ameliorating treatment of benign prostatic hypertrophy and/or lower urinary tract symptoms in a patient in need thereof. This aspect of the method includes administering to the patient an effective amount of a compound of Formula (I) or a form thereof in the form of a composition or medicament.

These and other aspects and advantages of the invention, which will become apparent in light of the detailed description below, are achieved through use of the compounds of this invention.

### DETAILED DESCRIPTION OF THE INVENTION

The present invention provides isoxazoline compounds of Formula (I) and a form thereof:

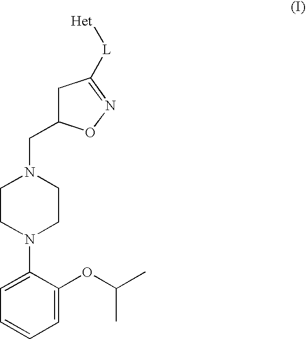

wherein
L is selected from the group consisting of —CH₂—, —CO—, and —CONH—;
Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with C₁₋₈alkyl, amino di-substituted with C₁₋₆alkyl, amino mono-substituted with ArC₁₋₁₆alkyl, amino disubstituted with ArC₁₋₁₆alkyl, Ar, Ar—CO, Ar-oxy, and Ar—SO₂;
Ar is selected from pyridinyl, phenyl and phenyl substituted with one or more substituents selected from halo, C₁₋₆alkyl, and C₁₋₆alkyloxy;
with the proviso that 1-[[4,5-dihydro-5-[[4-[2-(1-methylethoxy)phenyl]-1-piperazinyl]methyl]-3-isoxazolyl]methyl]-2-piperidinone Compound 1 is not included.

An example of the present invention is a compound of Formula (I) and a form thereof wherein Het is selected from the group consisting of pyrrolidinyl, piperidinyl, piperazinyl, 2,3-dihydro-1H-indolyl, 1,2,3,4-tetrahydro-quinolinyl, and morpholinyl.

An example of the present invention is a compound of Formula (I) and a form thereof wherein L is CH₂ or CO and wherein Het is connected to L via a nitrogen atom.

Another example of the present invention is a compound of Formula (I) and a form thereof wherein L is CONH and wherein Het is connected to L via a carbon atom.

An example of the present invention is a compound of Formula (I) and a form thereof wherein Het is selected from the group consisting of piperidin-2-on-1-yl, piperidin-1-yl, piperidin-1-yl substituted with phenylcarbonyl, 2,3-dihydroindol-1-yl, 2,3-dihydroindol-1-yl substituted with amino, 2,3-dihydroindol-1-yl substituted with difluorophenylmethylamino, 2,3-dihydroindol-1-yl substituted with monofluorophenylmethylamino, 2,3-dihydroindol-1-yl substituted with methoxyphenylmethylamino, 2,3-dihydroindol-1-yl substituted with nitro, 2,3-dihydroindol-1-yl substituted with bromo, 2,3-dihydroindol-1-yl substituted with chloro, piperazin-1-yl substituted with 2-(2-methylethyloxy), 5-bromo-(3,3-dimethyl)-2,3-dihydro-indol-1-yl, 5-fluoro-2,3-dihydro-indol-1-yl, 2,3-dihydro-indol-1-yl substituted with methyl, 2,3-dihydro-indolyl substituted with methylcarbonyl, 2,3-dihydro-indol-1-yl substituted with two methoxy substituents, piperidin-1-yl substituted with 4-chloro-phenyloxy, piperidin-1-yl substituted with 4-fluorophenylcarbonyl, 1,2,3,4-tetrahydro-quinolin-1-yl, piperidin-1-yl substituted with phenylmethyl, morpholin-4-yl, piperidin-1-yl substituted with phenyloxy, piperidin-1-yl substituted with phenyl sulphonyl, piperidin-1-yl substituted with two phenyls, piperidin-1-yl substituted with pyridin-4-yloxy, piperidin-1-yl substituted with phenyl substituted with methoxy, and pyrrolidin-1-yl.

An example of the present invention is a compound of Formula (I) and a form thereof wherein Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with ArC₁₋₆alkyl, Ar—CO, Ar-oxy, and Ar—SO₂.

An example of the present invention is a compound of Formula (I) and a form thereof wherein Het is selected from the group consisting of pyrrolidinyl, piperidinyl, 2,3-dihydro-1H-indolyl, 1,2,3,4-tetrahydro-quinolinyl, and morpholinyl.

An example of the present invention is a compound of Formula (I) and a form thereof wherein Het is selected from the group consisting of Het is selected from the group consisting of piperidin-2-on-1-yl, piperidin-1-yl, piperidin-1-yl substituted with phenylcarbonyl, 2,3-dihydroindol-1-yl, 2,3-dihydroindol-1-yl substituted with amino, 2,3-dihydroindol-1-yl substituted with difluorophenylmethylamino, 2,3-dihydroindol-1-yl substituted with monofluorophenylmethylamino, 2,3-dihydroindol-1-yl substituted with nitro, 2,3-dihydroindol-1-yl substituted with bromo, 2,3-dihydroindol-1-yl substituted with chloro, 5-bromo-(3,3-dimethyl)-2,3-dihydro-indol-1-yl, 5-fluoro-2,3-dihydro-indol-1-yl, 2,3-dihydro-indol-1-yl substituted with methyl, 2,3-dihydro-indolyl substituted with methylcarbonyl, 2,3-dihydro-indol-1-yl substituted with two methoxy substituents, piperidin-1-yl substituted with 4-fluorophenylcarbonyl, 1,2,3,4-tetrahydro-quinolin-1-yl, morpholin-4-yl, piperidin-1-yl substituted with phenyloxy, piperidin-1-yl substituted with phenyl sulphonyl and pyrrolidin-1-yl.

An embodiment of the invention is a compound of Formula (I) selected from the group consisting of:

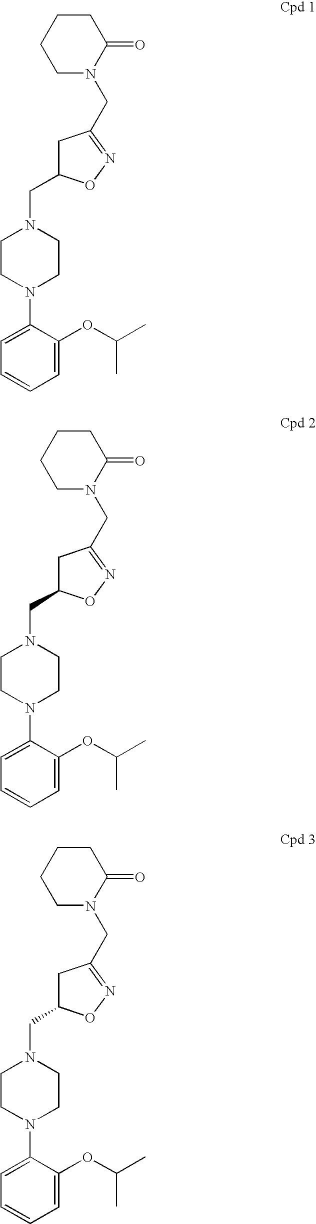

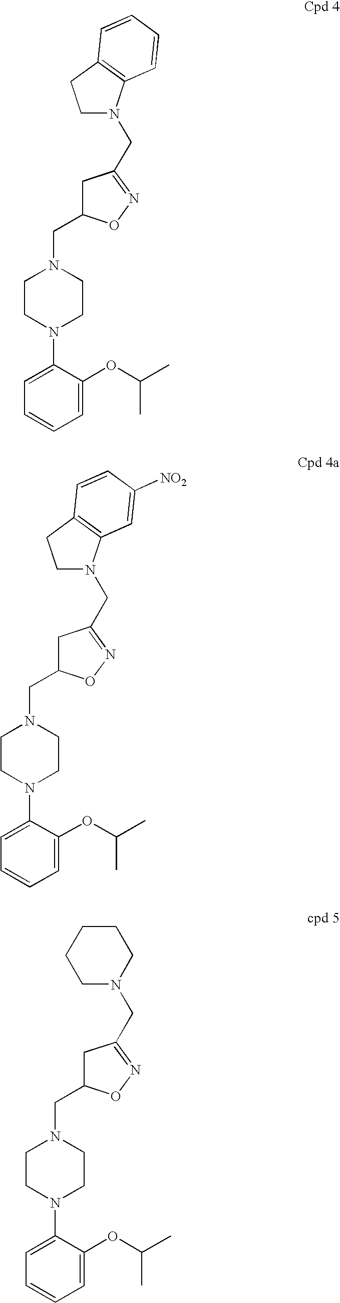

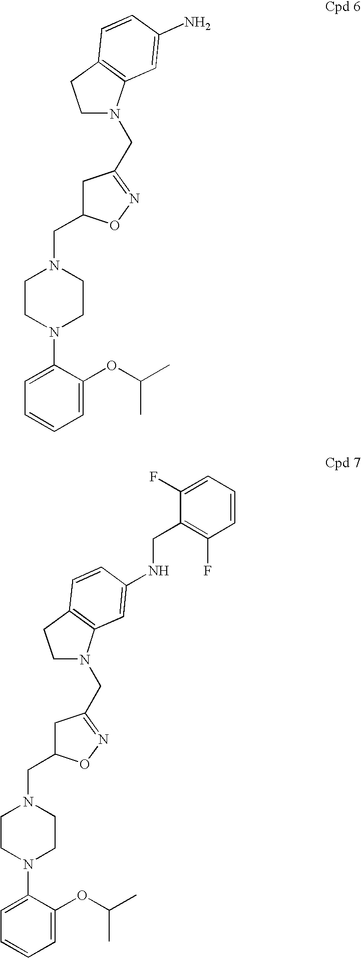

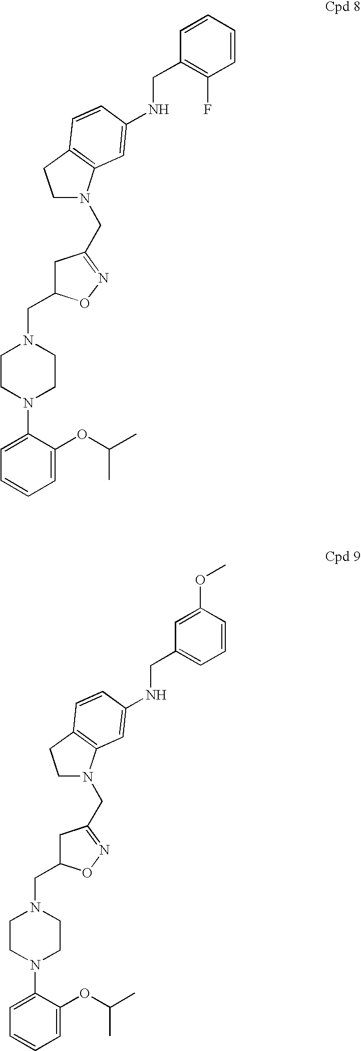

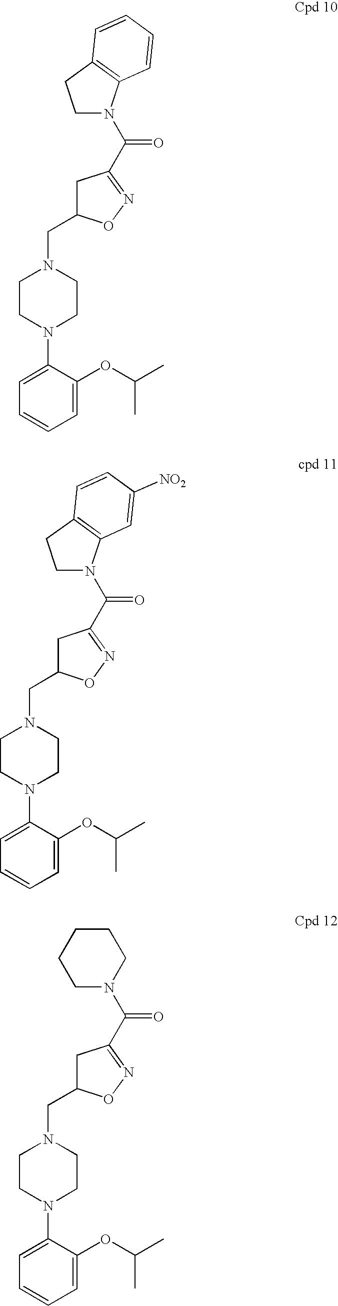

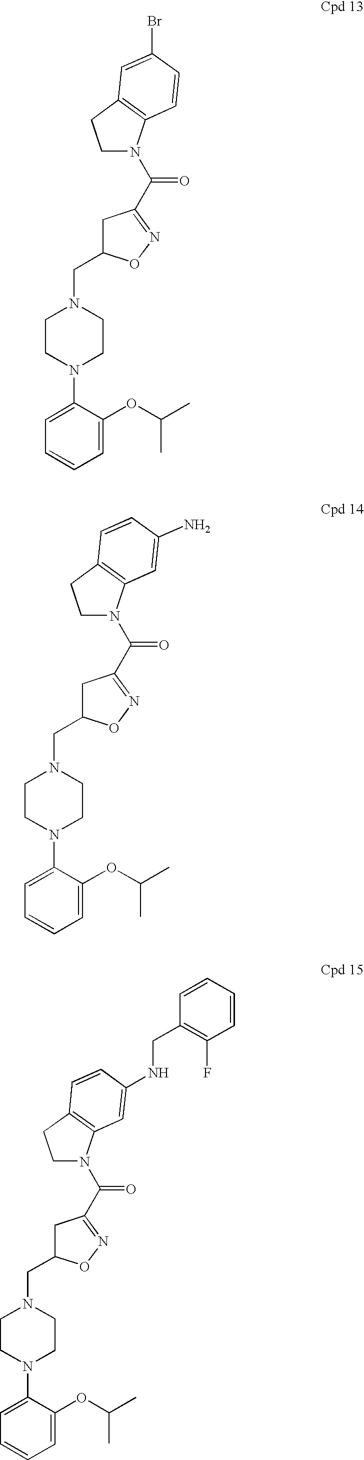

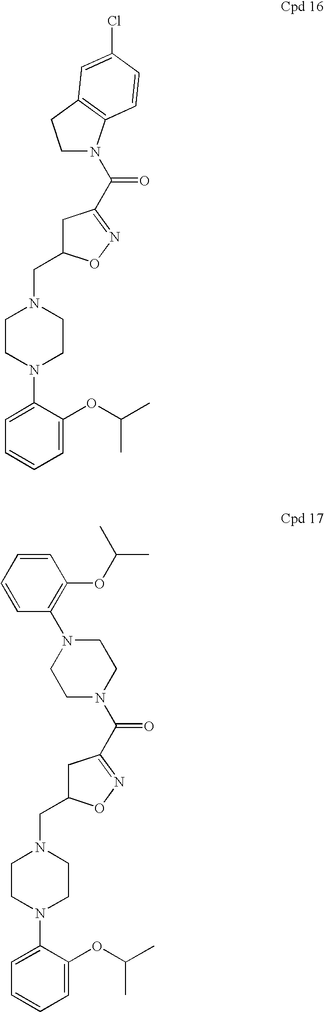

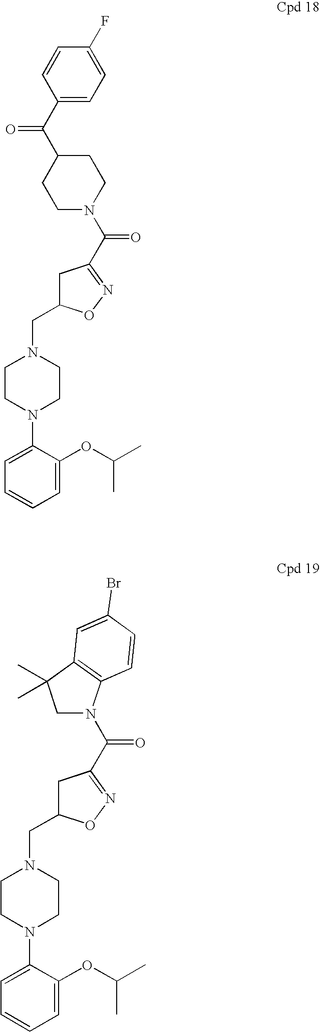

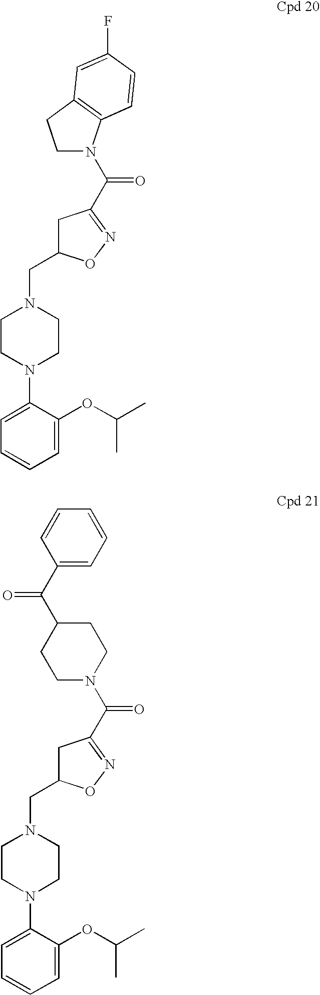

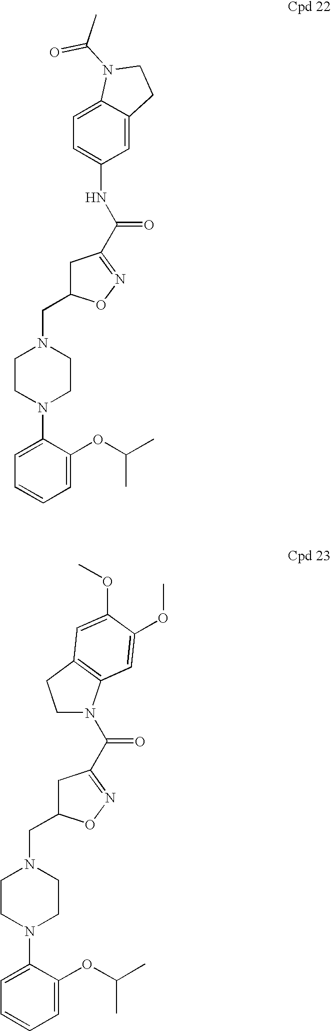

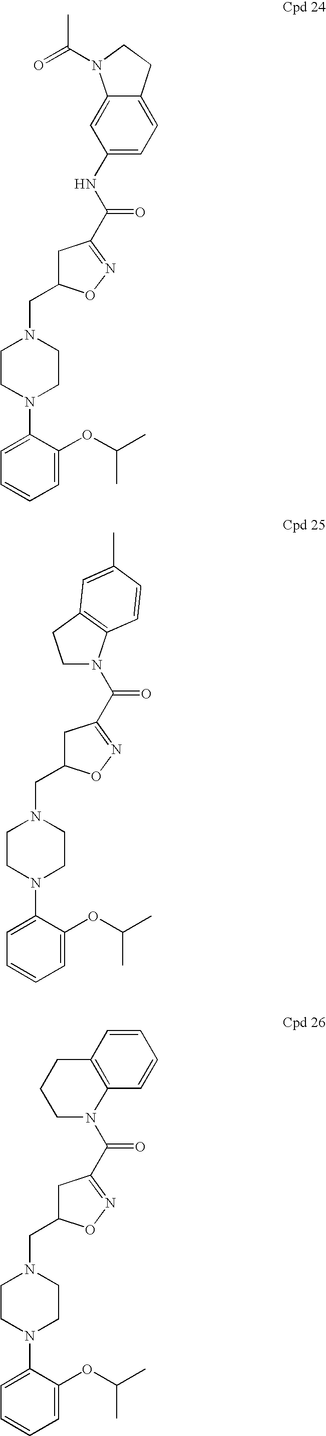

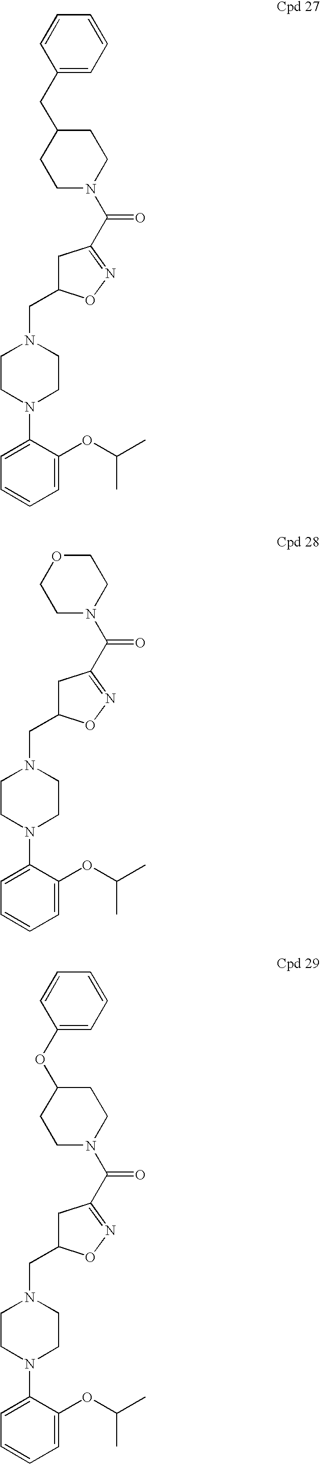

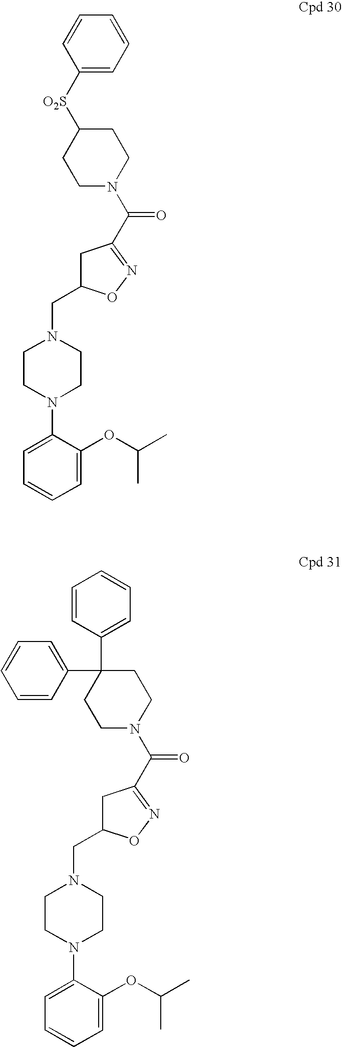

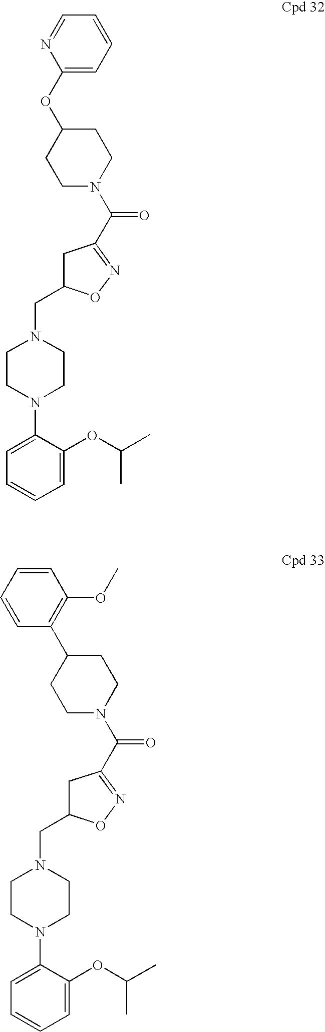

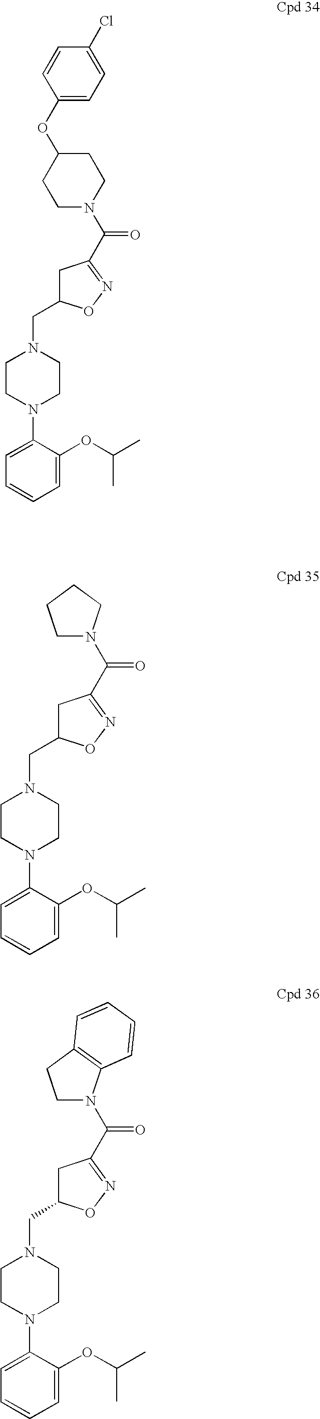

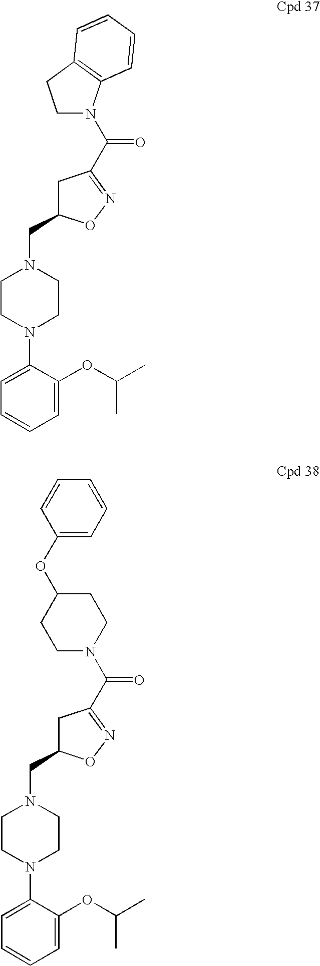

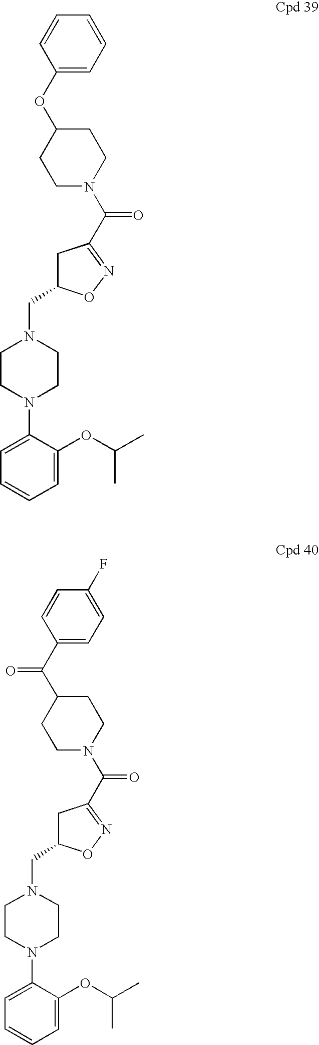

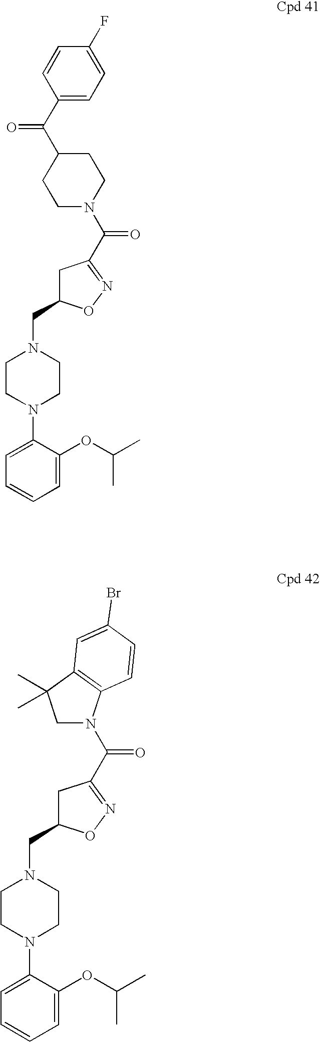

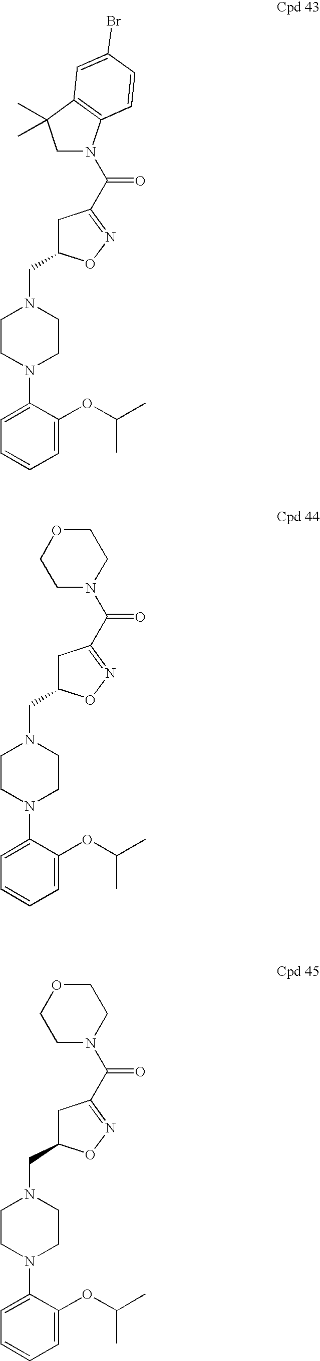

An embodiment of the present invention includes compounds of Formula (I) selected from the group consisting of:

| Cpd | Name |
|---|---|
| 1 | 1-{5-[4-(2-isopropoxy-phenyl)-piperazin-1-ylmethyl]-4,5-dihydro- |
| | isoxazol-3-ylmethyl}-piperidin-2-one, |
| 2 | 1-{(5R*)-5-[4-(2-isopropoxy-phenyl)-piperazin-1-ylmethyl]-4,5-dihydro- |
| | isoxazol-3-ylmethyl}-piperidin-2-one, |
| 3 | 1-{(5S*)-5-[4-(2-isopropoxy-phenyl)-piperazin-1-ylmethyl]-4,5-dihydro- |
| | isoxazol-3-ylmethyl}-piperidin-2-one, |
| 4 | 1-{5-[4-(2-isopropoxy-phenyl)-piperazin-1-ylmethyl]-4,5-dihydro- |
| | isoxazol-3-ylmethyl}-2,3-dihydro-1H-indole, |
| 4a | 1-{5-[4-(2-isopropoxy-phenyl)-piperazin-1-ylmethyl]-4,5-dihydro- |
| | isoxazol-3-ylmethyl}-6-nitro-2,3-dihydro-1H-indole, |
| 5 | 1-(2-isopropoxy-phenyl)-4-(3-piperidin-1-ylmethyl-4,5-dihydro-isoxazol- |
| | 5-ylmethyl)-piperazine, |
| 6 | 1-{5-[4-(2-isopropoxy-phenyl)-piperazin-1-ylmethyl]-4,5-dihydro- |
| | isoxazol-3-ylmethyl}-2,3-dihydro-1H-indol-6-ylamine, |
| 7 | (2,6-difluoro-benzyl)-(1-{5-[4-(2-isopropoxy-phenyl)-piperazin-1- |
| | ylmethyl]-4,5-dihydro-isoxazol-3-ylmethyl}-2,3-dihydro-1H-indol-6-yl)-amine, |
| 8 | (2-fluoro-benzyl)-(1-{5-[4-(2-isopropoxy-phenyl)-piperazin-1-ylmethyl]- |
| | 4,5-dihydro-isoxazol-3-ylmethyl}-2,3-dihydro-1H-indol-6-yl)-amine, |
| 9 | (1-{5-[4-(2-isopropoxy-phenyl)-piperazin-1-ylmethyl]-4,5-dihydro- |
| | isoxazol-3-ylmethyl}-2,3-dihydro-1H-indol-6-yl)-(3-methoxy-benzyl)-amine, |
| 10 | (2,3-dihydro-indol-1-yl)-{5-[4-(2-isopropoxy-phenyl)-piperazin-1- |
| | ylmethyl]-4,5-dihydro-isoxazol-3-yl}-methanone, |
| 11 | {5-[4-(2-isopropoxy-phenyl)-piperazin-1-ylmethyl]-4,5-dihydro- |
| | isoxazol-3-yl}-(6-nitro-2,3-dihydro-indol-1-yl)-methanone, |
| 12 | {5-[4-(2-isopropoxy-phenyl)-piperazin-1-ylmethyl]-4,5-dihydro- |
| | isoxazol-3-yl}-piperidin-1-yl-methanone, |
| 13 | (5-bromo-2,3-dihydro-indol-1-yl)-{5-[4-(2-isopropoxy-phenyl)- |
| | piperazin-1-ylmethyl]-4,5-dihydro-isoxazol-3-yl}-methanone, |
| 14 | (6-amino-2,3-dihydro-indol-1-yl)-{5-[4-(2-isopropoxy-phenyl)- |
| | piperazin-1-ylmethyl]-4,5-dihydro-isoxazol-3yl}-methanone, |
| 15 | [6-(2-fluoro-benzylamino)-2,3-dihydro-indol-1-yl]-{5-[4-(2-isopropoxy- |
| | phenyl)-piperazin-1-ylmethyl]-4,5-dihydro-isoxazol-3-yl}-methanone, |
| 16 | (5-chloro-2,3-dihydro-indol-1-yl)-{5-[4-(2-isopropoxy-phenyl)- |
| | piperazin-1-ylmethyl]-4,5-dihydro-isoxazol-3-yl}-methanone, |
| 17 | [4-(2-isopropoxy-phenyl)-piperazin-1-yl]-{5-[4-(2-isopropoxy-phenyl)- |
| | piperazin-1-ylmethyl]-4,5-dihydro-isoxazol-3-yl}-methanone, |
| 18 | [4-(4-fluoro-benzoyl)-piperidin-1-yl]-{5-[4-(2-isopropoxy-phenyl)- |
| | piperazin-1-ylmethyl]-4,5-dihydro-isoxazol-3-yl}-methanone, |
| 19 | (5-bromo-3,3-dimethyl-2,3-dihydro-indol-1-yl)-{5-[4-(2-isopropoxy- |
| | phenyl)-piperazin-1-ylmethyl]-4,5-dihydro-isoxazol-3-yl}-methanone, |
| 20 | (5-fluoro-2,3-dihydro-indol-1-yl)-{5-[4-(2-isopropoxy-phenyl)-piperazin- |
| | 1-ylmethyl]-4,5-dihydro-isoxazol-3-yl}-methanone, |
| 21 | (4-benzoyl-piperidin-1-yl)-{5-[4-(2-isopropoxy-phenyl)-piperazin-1- |
| | ylmethyl]-4,5-dihydro-isoxazol-3-yl}-methanone, |
| 22 | 5-[4-(2-isopropoxy-phenyl)-piperazin-1-ylmethyl]-4,5-dihydro- |
| | isoxazole-3-carboxylic acid (1-acetyl-2,3-dihydro-1H-indol-5-yl)-amide, |
| 23 | (5,6-dimethoxy-2,3-dihydro-indol-1-yl)-{5-[4-(2-isopropoxy-phenyl)- |
| | piperazin-1-ylmethyl]-4,5-dihydro-isoxazol-3-yl}-methanone, |
| 24 | 5-[4-(2-isopropoxy-phenyl)-piperazin-1-ylmethyl]-4,5-dihydro- |
| | isoxazole-3-carboxylic acid (1-acetyl-2,3-dihydro-1H-indol-6-yl)-amide, |
| 25 | {5-[4-(2-isopropoxy-phenyl)-piperazin-1-ylmethyl]-4,5-dihydro- |
| | isoxazol-3-yl}-(5-methyl-2,3-dihydro-indol-1-yl)-methanone, |
| 26 | (3,4-dihydro-2H-quinolin-1-yl)-{5-[4-(2-isopropoxy-phenyl)-piperazin- |
| | 1-ylmethyl]-4,5-dihydro-isoxazol-3-yl}-methanone, |
| 27 | (4-benzyl-piperidin-1-yl)-{5-[4-(2-isopropoxy-phenyl)-piperazin-1- |
| | ylmethyl]-4,5-dihydro-isoxazol-3-yl}-methanone, |
| 28 | {5-[4-(2-isopropoxy-phenyl)-piperazin-1-ylmethyl]-4,5-dihydro- |
| | isoxazol-3-yl}-morpholin-4-yl-methanone, |
| 29 | {5-[4-(2-isopropoxy-phenyl)-piperazin-1-ylmethyl]-4,5-dihydro- |
| | isoxazol-3-yl}-(4-phenoxy-piperidin-1-yl)-methanone, |
| 30 | (4-benzenesulfonyl-piperidin-1-yl)-{5-[4-(2-isopropoxy-phenyl)- |
| | piperazin-1-ylmethyl]-4,5-dihydro-isoxazol-3-yl}-methanone, |
| 31 | (4,4-diphenyl-piperidin-1-yl)-{5-[4-(2-isopropoxy-phenyl)-piperazin-1- |
| | ylmethyl]-4,5-dihydro-isoxazol-3-yl}-methanone, |
| 32 | {5-[4-(2-isopropoxy-phenyl)-piperazin-1-ylmethyl]-4,5-dihydro- |
| | isoxazol-3-yl}-[4-(pyridin-2-yloxy)-piperidin-1-yl]-methanone, |
| 33 | {5-[4-(2-isopropoxy-phenyl)-piperazin-1-ylmethyl]-4,5-dihydro- |
| | isoxazol-3-yl}-[4-(2-methoxy-phenyl)-piperidin-1-yl]-methanone, |
| 34 | [4-(4-chloro-phenoxy)-piperidin-1-yl]-{5-[4-(2-isopropoxy-phenyl)- |
| | piperazin-1-ylmethyl]-4,5-dihydro-isoxazol-3-yl}-methanone, |
| 35 | {5-[4-(2-isopropoxy-phenyl)-piperazin-1-ylmethyl]-4,5-dihydro- |
| | isoxazol-3-yl}-pyrrolidin-1-yl-methanone, |
| 36 | (2,3-dihydro-indol-1-yl)-{(5S*)-5-[4-(2-isopropoxy-phenyl)-piperazin-1- |
| | ylmethyl]-4,5-dihydro-isoxazol-3-yl}-methanone, |
| 37 | (2,3-dihydro-indol-1-yl)-{(5R*)-5-[4-(2-isopropoxy-phenyl)-piperazin-1- |
| | ylmethyl]-4,5-dihydro-isoxazol-3-yl}-methanone, |
| 38 | {(5R*)-5-[4-(2-isopropoxy-phenyl)-piperazin-1-ylmethyl]-4,5-dihydro- |
| | isoxazol-3-yl}-(4-phenoxy-piperidin-1-yl)-methanone, |
| 39 | {(5S*)-5-[4-(2-isopropoxy-phenyl)-piperazin-1-ylmethyl]-4,5-dihydro- |
| | isoxazol-3-yl}-(4-phenoxy-piperidin-1-yl)-methanone, |
| 40 | [4-(4-fluoro-benzoyl)-piperidin-1-yl]-{(5S*)-5-[4-(2-isopropoxy-phenyl)- |
| | piperazin-1-ylmethyl]-4,5-dihydro-isoxazol-3-yl}-methanone, |
| 41 | [4-(4-fluoro-benzoyl)-piperidin-1-yl]-{(5R*)-5-[4-(2-isopropoxy- |
| | phenyl)-piperazin-1-ylmethyl]-4,5-dihydro-isoxazol-3-yl}-methanone, |
| 42 | (5-bromo-3,3-dimethyl-2,3-dihydro-indol-1-yl)-{(5R*)-5-[4-(2- |
| | isopropoxy-phenyl)-piperazin-1-ylmethyl]-4,5-dihydro-isoxazol-3-yl}-methanone, |
| 43 | (5-bromo-3,3-dimethyl-2,3-dihydro-indol-1-yl)-{(5S*)-5-[4-(2- |
| | isopropoxy-phenyl)-piperazin-1-ylmethyl]-4,5-dihydro-isoxazol-3-yl}-methanone, |
| 44 | {(5S*)-5-[4-(2-isopropoxy-phenyl)-piperazin-1-ylmethyl]-4,5-dihydro- |
| | isoxazol-3-yl}-morpholin-4-yl-methanone, and |
| 45 | {(5R*)-5-[4-(2-Isopropoxy-phenyl)-piperazin-1-ylmethyl]-4,5-dihydro- |
| | isoxazol-3-yl}-morpholin-4-yl-methanone. |

An embodiment of the present invention includes compounds of Formula (I) selected from the group consisting of:

| Cpd | Name |
|---|---|
| 1 | 1-{5-[4-(2-isopropoxy-phenyl)-piperazin-1-ylmethyl]-4,5-dihydro-isoxazol-3- |
| | ylmethyl}-piperidin-2-one, |
| 2 | 1-{(5R*)-5-[4-(2-isopropoxy-phenyl)-piperazin-1-ylmethyl]-4,5-dihydro- |
| | isoxazol-3-ylmethyl}-piperidin-2-one, |
| 3 | 1-{(5S*)-5-[4-(2-isopropoxy-phenyl)-piperazin-1-ylmethyl]-4,5-dihydro- |
| | isoxazol-3-ylmethyl}-piperidin-2-one, |
| 4 | 1-{5-[4-(2-isopropoxy-phenyl)-piperazin-1-ylmethyl]-4,5-dihydro- |
| | isoxazol-3-ylmethyl}-2,3-dihydro-1H-indole, |
| 4a | 1-{5-[4-(2-isopropoxy-phenyl)-piperazin-1-ylmethyl]-4,5-dihydro- |
| | isoxazol-3-ylmethyl}-6-nitro-2,3-dihydro-1H-indole, |
| 6 | 1-{5-[4-(2-isopropoxy-phenyl)-piperazin-1-ylmethyl]-4,5-dihydro- |
| | isoxazol-3-ylmethyl}-2,3-dihydro-1H-indol-6-ylamine, |
| 7 | (2,6-difluoro-benzyl)-(1-{5-[4-(2-isopropoxy-phenyl)-piperazin-1- |
| | ylmethyl]-4,5-dihydro-isoxazol-3-ylmethyl}-2,3-dihydro-1H-indol-6-yl)-amine, |
| 8 | (2-fluoro-benzyl)-(1-{5-[4-(2-isopropoxy-phenyl)-piperazin-1-ylmethyl]-4,5- |
| | dihydro-isoxazol-3-ylmethyl}-2,3-dihydro-1H-indol-6-yl)-amine, |
| 10 | (2,3-dihydro-indol-1-yl)-{5-[4-(2-isopropoxy-phenyl)-piperazin-1- |
| | ylmethyl]-4,5-dihydro-isoxazol-3-yl}-methanone, |
| 11 | {5-[4-(2-isopropoxy-phenyl)-piperazin-1-ylmethyl]-4,5-dihydro- |
| | isoxazol-3-yl}-(6-nitro-2,3-dihydro-indol-1-yl)-methanone, |
| 12 | {5-[4-(2-isopropoxy-phenyl)-piperazin-1-ylmethyl]-4,5-dihydro- |
| | isoxazol-3-yl}-piperidin-1-yl-methanone, |
| 13 | (5-bromo-2,3-dihydro-indol-1-yl)-{5-[4-(2-isopropoxy-phenyl)- |
| | piperazin-1-ylmethyl]-4,5-dihydro-isoxazol-3-yl}-methanone, |
| 14 | (6-amino-2,3-dihydro-indol-1-yl)-{5-[4-(2-isopropoxy-phenyl)- |
| | piperazin-1-ylmethyl]-4,5-dihydro-isoxazol-3yl}-methanone, |
| 15 | [6-(2-fluoro-benzylamino)-2,3-dihydro-indol-1-yl]-{5-[4-(2-isopropoxy- |
| | phenyl)-piperazin-1-ylmethyl]-4,5-dihydro-isoxazol-3-yl}-methanone, |
| 16 | (5-chloro-2,3-dihydro-indol-1-yl)-{5-[4-(2-isopropoxy-phenyl)- |
| | piperazin-1-ylmethyl]-4,5-dihydro-isoxazol-3-yl}-methanone, |
| 18 | [4-(4-fluoro-benzoyl)-piperidin-1-yl]-{5-[4-(2-isopropoxy-phenyl)- |
| | piperazin-1-ylmethyl]-4,5-dihydro-isoxazol-3-yl}-methanone, |
| 19 | (5-bromo-3,3-dimethyl-2,3-dihydro-indol-1-yl)-{5-[4-(2-isopropoxy- |
| | phenyl)-piperazin-1-ylmethyl]-4,5-dihydro-isoxazol-3-yl}-methanone, |
| 20 | (5-fluoro-2,3-dihydro-indol-1-yl)-{5-[4-(2-isopropoxy-phenyl)-piperazin- |
| | 1-ylmethyl]-4,5-dihydro-isoxazol-3-yl}-methanone, |
| 21 | (4-benzoyl-piperidin-1-yl)-{5-[4-(2-isopropoxy-phenyl)-piperazin-1- |
| | ylmethyl]-4,5-dihydro-isoxazol-3-yl}-methanone, |
| 22 | 5-[4-(2-isopropoxy-phenyl)-piperazin-1-ylmethyl]-4,5-dihydro- |
| | isoxazole-3-carboxylic acid (1-acetyl-2,3-dihydro-1H-indol-5-yl)-amide, |
| 23 | (5,6-dimethoxy-2,3-dihydro-indol-1-yl)-{5-[4-(2-isopropoxy-phenyl)- |
| | piperazin-1-ylmethyl]-4,5-dihydro-isoxazol-3-yl}-methanone, |
| 24 | 5-[4-(2-isopropoxy-phenyl)-piperazin-1-ylmethyl]-4,5-dihydro- |
| | isoxazole-3-carboxylic acid (1-acetyl-2,3-dihydro-1H-indol-6-yl)-amide, |
| 25 | {5-[4-(2-isopropoxy-phenyl)-piperazin-1-ylmethyl]-4,5-dihydro- |
| | isoxazol-3-yl}-(5-methyl-2,3-dihydro-indol-1-yl)-methanone, |
| 26 | (3,4-dihydro-2H-quinolin-1-yl)-{5-[4-(2-isopropoxy-phenyl)-piperazin- |
| | 1-ylmethyl]-4,5-dihydro-isoxazol-3-yl}-methanone, |
| 28 | {5-[4-(2-isopropoxy-phenyl)-piperazin-1-ylmethyl]-4,5-dihydro- |
| | isoxazol-3-yl}-morpholin-4-yl-methanone, |
| 29 | {5-[4-(2-isopropoxy-phenyl)-piperazin-1-ylmethyl]-4,5-dihydro- |
| | isoxazol-3-yl}-(4-phenoxy-piperidin-1-yl)-methanone, |
| 30 | (4-benzenesulfonyl-piperidin-1-yl)-{5-[4-(2-isopropoxy-phenyl)- |
| | piperazin-1-ylmethyl]-4,5-dihydro-isoxazol-3-yl}-methanone, |
| 35 | {5-[4-(2-isopropoxy-phenyl)-piperazin-1-ylmethyl]-4,5-dihydro- |
| | isoxazol-3-yl}-pyrrolidin-1-yl-methanone, |
| 37 | (2,3-dihydro-indol-1-yl)-{(5R*)-5-[4-(2-isopropoxy-phenyl)-piperazin-1- |
| | ylmethyl]-4,5-dihydro-isoxazol-3-yl}-methanone, |
| 39 | {(5S*)-5-[4-(2-isopropoxy-phenyl)-piperazin-1-ylmethyl]-4,5-dihydro- |
| | isoxazol-3-yl}-(4-phenoxy-piperidin-1-yl)-methanone, |
| 40 | [4-(4-fluoro-benzoyl)-piperidin-1-yl]-{(5S*)-5-[4-(2-isopropoxy-phenyl)- |
| | piperazin-1-ylmethyl]-4,5-dihydro-isoxazol-3-yl}-methanone, |
| 42 | (5-bromo-3,3-dimethyl-2,3-dihydro-indol-1-yl)-{(5R*)-5-[4-(2- |
| | isopropoxy-phenyl)-piperazin-1-ylmethyl]-4,5-dihydro-isoxazol-3-yl}-methanone, and |
| 44 | {(5S*)-5-[4-(2-isopropoxy-phenyl)-piperazin-1-ylmethyl]-4,5-dihydro-isoxazol- |
| | 3-yl}-morpholin-4-yl-methanone. |

### Compound Forms

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof. Preferably the term “form” refers to a salt, a stereoisomer, a tautomer, or a solvate.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Suitable salt forms include acid addition salts which may, for example, be formed by mixing a solution of the compound according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulphuric acid, tartaric acid, trifluoroacetic acid and the like.

Furthermore when the compounds of the present invention carry an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts include the following: acetate, adipate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art. The scope of the present invention encompasses all such protected compound forms and mixtures thereof.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “stereoisomer” refers to an isomer that has the same molecular formula and the same sequence of covalently bonded atoms but a different spatial orientation.

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of the isolated species rotates the plane of polarized light in the opposite direction such that the mixture is devoid of optical activity.

The term “enantiomer” means an isomer having a non-superimposable mirror image. The term “diastereomer” means stereoisomers that are not enantiomers.

The term “chiral” means a molecule that, in a given configuration, cannot be superimposed on its mirror image. This is in contrast to achiral molecules that can be superimposed on their mirror images.

The symbols “R” and “S” represent the configuration of atoms around a stereogenic carbon atom. The symbols “R*” and “S*” represent the relative configuration of atoms around a stereogenic carbon atom. The isomeric descriptors (“R.” “S,” “R*” “S*”) are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the present invention may have one or more polymorph or amorphous crystalline forms and, as such, are intended to be included in the scope of the invention. In addition, some of the compounds may form solvates with water (i.e., hydrates) or common organic solvents (e.g., organic esters such as ethanolate and the like) and, as such, are also intended to be encompassed within the scope of this invention.

### Chemical Definitions

As used herein, the following terms are intended to have the following meanings (additional definitions are provided where needed throughout the Specification). The definitions herein may specify that a chemical term has an indicated formula. The particular formula provided is not intended to limit the scope of the invention, but is provided as an illustration of the term. The scope of the per se definition of the term is intended to include the plurality of variations expected to be included by one of ordinary skill in the art.

The term “C₁₋₆ alkyl,” whether used alone or as part of a substituent group, means a straight or branched chain monovalent hydrocarbon alkyl radical or alkyldiyl linking group comprising from 1 to 6 carbon atoms, wherein the radical is derived by the removal of one hydrogen atom from a single carbon atom and the alkyldiyl linking group is derived by the removal of one hydrogen atom from each of two carbon atoms in the chain, such as, for example methyl, ethyl, 1-propyl, 2-propyl, 1-butyl, 2-butyl, tertiary butyl, 1-pentyl, 2-pentyl, 3-pentyl, 1-hexyl, 2-hexyl, 3-hexyl, and the like. Examples include C₁₋₄alkyl groups.

The term “C₁₋₆ alkoxy,” whether used alone or as part of a substituent group, refers to an alkyl or alkyldiyl radical attached through an oxygen-linking atom. Typical alkoxy groups comprising from 1 to 6 carbon atoms, such as, for example, methoxy, ethoxy, propoxy, butoxy, pentoxy, hexoxy and the like. An alkoxy radical may be attached to a core molecule and further substituted where indicated. Examples include C₁₋₄alkoxy groups.

The term “heterocyclyl,” whether used alone or as part of a substituent group, refers to a saturated or partially unsaturated monocyclic or polycyclic ring radical derived by the removal of one hydrogen atom from a single carbon or nitrogen ring atom. Typical heterocyclyl radicals include pyrrolidinyl, piperidinyl, morpholinyl, thiomorpholinyl, piperazinyl, 2,3-dihydro-1H-indolyl, 1,2,3,4-tetrahydro-quinolinyl.

In an example of the invention the heterocyclyl radicals are selected from the group (in these structures the (L)-represents the connection to the rest of the molecule; when a hydrogen is shown on a nitrogen then that hydrogen is there to complete valency but this hydrogen can be substituted by another radical) such as:

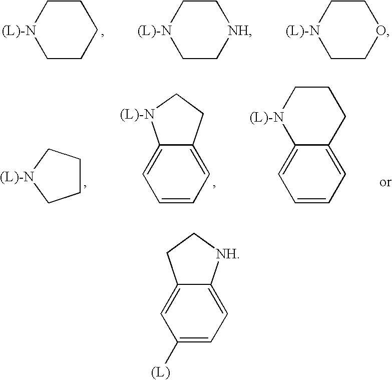

The term “hetero” used as a prefix for a ring system refers to the replacement of at least one ring carbon atom with one or more heteroatoms independently selected from N, S, or O. Examples include rings wherein 1, 2, 3 or 4 ring members are a nitrogen atom; or, 0, 1, 2 or 3 ring members are nitrogen atoms and 1 member is an oxygen or sulfur atom. When allowed by available valences, up to two adjacent ring members may be heteroatoms; wherein one heteroatom is nitrogen and the other is one heteroatom selected from N, S or O.

The term “aromatic” refers to a cycloalkyl hydrocarbon ring system having an unsaturated, conjugated π electron system.

The term “halogen” includes fluoro, chloro, bromo, and iodo.

The term “oxo” refers to a carbonyl bond, preferably a keto form. For instance, piperidinyl substituted with “oxo” refers to, for example:

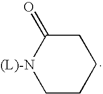

The term “substituted,” refers to a core molecule on which one or more hydrogen atoms have been replaced with one or more functional radical moieties. The number that is allowed by available valences limits the amount of substituents. Substitution is not limited to the core molecule, but may also occur on a substituent radical, whereby the substituent radical becomes a linking group.

The term “optionally substituted” means that the radical or substituent that is “optionally substituted” is either unsubstituted or substituted.

The term “independently selected” refers to one or more substituents selected from a group of substituents variable group, wherein the selected substituents may be the same or different.

The term “phenyloxy” can be interchangeably used with “phenoxy” and is

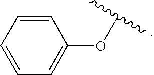

defined as

### Therapeutic Use

The ability of compounds of the present invention to specifically bind to the α₁ₐ as well as to the α_{1d} receptor makes them useful for the treatment of BPH. The specificity of binding of compounds showing affinity for the α₁ₐ and the α_{1d} receptor is compared against the binding affinities to other types of alpha receptors.

An aspect of the present invention includes a compound of Formula (I) having an IC₅₀ (50% inhibition concentration) against the activity of either or both the α₁ₐ and/or α_{1d} adrenoreceptor in a range of about 25 μM or less, of about 10 μM or less, of about 1 μM or less, of about 0.5 μM or less, of about 0.25 μM or less or of about 0.1 μM or less.

Another aspect of the present invention includes dual selective α₁ₐ/α_{1d} adrenoreceptor antagonists for treating, ameliorating or preventing a plurality of α₁ₐ and/or α_{1d} adrenoreceptor mediated disorders or diseases.

The usefulness of a compound of the present invention or composition thereof as a dual selective α₁ₐ/α_{1d} adrenoreceptor antagonist can be determined according to the methods disclosed herein. The scope of such use includes the treatment of benign prostatic hypertrophy and/or lower urinary tract symptoms.

An aspect of the use for a compound of Formula (I) includes use of an instant compound as a marker, wherein the compound is labeled with a ligand such as a radioligand (selected from deuterium, tritium and the like).

The present invention is also directed to a method for treating, ameliorating or preventing an α₁ₐ and/or α_{1d} adrenoreceptor mediated disorder or disease in a subject in need of such treatment, amelioration or prevention comprising administering to the subject a therapeutically or prophylactically effective amount of a compound of Formula (I) or a form or composition thereof.

An aspect of the method of the present invention further includes treating Benign Prostatic Hyperplasia in a subject in need of such treatment comprising administering to the subject in need of such treatment a therapeutically effective amount of a compound of Formula (I) or a form or composition thereof.

An aspect of the method of the present invention further includes treating Lower Urinary Tract Symptoms in a subject in need of such treatment comprising administering to the subject in need of such treatment a therapeutically effective amount of a compound of Formula (I) or a form or composition thereof.

The present invention is also directed to a method for treating Benign Prostatic Hyperplasia in a subject in need of such treatment comprising administering to the subject in need of such treatment a therapeutically effective amount of 1-{5-[4-(2-isopropoxy-phenyl)-piperazin-1-ylmethyl]-4,5-dihydro-isoxazol-3-ylmethyl}-piperidin-2-one.

The present invention is also directed to a method for treating Lower Urinary Tract Symptoms in a subject in need of such treatment comprising administering to the subject in need of such treatment a therapeutically effective amount of 1-{5-[4-(2-isopropoxy-phenyl)-piperazin-1-ylmethyl]-4,5-dihydro-isoxazol-3-ylmethyl}-piperidin-2-one.

Another aspect of the method of the present invention further includes administering to the subject an effective amount of a compound of Formula (I) or composition thereof in the form of a medicament. Consequently, the invention encompasses the use of the compound of Formula (I) as a medicament.

Accordingly, the present invention includes the use of a compound of Formula (I) for the manufacture of a medicament for treating any of the diseases, disorders or conditions mentioned in any of the foregoing methods.

The term “subject” as used herein, refers to an animal, preferably a mammal, most preferably a human, which has been a patient or the object of treatment, prevention, observation or experiment.

The term “administering” is to be interpreted liberally in accordance with the methods of the present invention. Such methods include therapeutically or prophylactically administering an effective amount of a composition or medicament of the present invention at different times during the course of a therapy or concurrently in a combination form. Prophylactic administration can occur prior to the manifestation of symptoms characteristic of a α₁ₐ and/or α_{1d} adrenoreceptor mediated disorder or disease such that the disorder or disease is treated, ameliorated, prevented or otherwise delayed in its progression. The methods of the present invention are further to be understood as embracing all therapeutic or prophylactic treatment regimens used by those skilled in the art.

The terms “therapeutically effective amount” or “prophylactically effective amount” refer to that amount of active compound or pharmaceutical agent that elicits the biological or medicinal response in a tissue system, animal or human, that is being sought by a researcher, veterinarian, medical doctor, or other clinician, which includes alleviation of the symptoms of the syndrome, disorder or disease being treated.

The effective amount of a compound of Formula (I) exemplified in a method of the present invention is in a range of from about 0.001 mg/kg/day to about 300 mg/kg/day.

The term “medicament” refers to a product for use in treating, preventing or ameliorating a disease, disorder or condition as described herein.

Wherein the present invention is directed to the administration of a combination of a compound of Formula (I) and another agent for the treatment of BPH, the terms “therapeutically effective amount” or “prophylactically effective amount” shall mean that amount of the combination of agents taken together so that the combined effect elicits the desired biological or medicinal response.

Representative compounds of the present invention exhibit high selectivity for the α₁ₐ and α_{1d} adrenergic receptor. Moreover representative compounds of the present invention show low to very low affinity for the α_{1d} receptor. As a consequence thereof, the compounds of the present invention are believed to lower the intraurethral pressure without the unwanted side effects.

These compounds can be administered in dosages effective to antagonize the α₁ₐ and α_{1d} receptor where such treatment is needed, as in BHP.

### Pharmaceutical Compositions

The present invention also has the objective of providing suitable topical, oral, systemic and parenteral pharmaceutical formulations for use in the novel methods of treatment of the present invention. The compositions containing compounds of this invention as the active ingredient for use in the specific antagonism of human α₁ₐ adrenergic receptors can be administered in a wide variety of therapeutic dosage forms in conventional vehicles for systemic administration.

The present invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto injector devices or suppositories; for oral, parenteral, intranasal, sublingual or rectal administration, or for administration by inhalation or insufflation.

Alternatively, the compositions may be presented in a form suitable for once-weekly or once-monthly administration; for example, an insoluble salt of the active compound, such as the decanoate salt, may be adapted to provide a depot preparation for intramuscular injection.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogenous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. An enteric layer can separate the two components. That enteric layer serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethyl cellulose, methylcellulose, polyvinyl-pyrrolidone or gelatin.

As used herein, the term “composition” is intended to encompass a product comprising the specified ingredients in the specified amounts, as well as any product which results, directly or indirectly, from combination of the specified ingredients in the specified amounts.

An effective but non-toxic amount of the compound desired can be employed as a α₁ₐ/α_{1d} antagonistic agent. Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

Compounds of this invention may be administered in any of the foregoing compositions and according to dosage regimens established in the art whenever specific blockade of the human alpha-adrenergic receptor is required.

The daily dosage of the products may be varied over a wide range from 0.001 to 3,000 mg per adult human per day. For oral administration, the compositions are preferably provided in the form of tablets containing 0.01, 0.05, 0.1, 0.5, 1.0, 2.5, 5.0, 10.0, 15.0, 25.0, 50.0 and milligrams of the active ingredient for the symptomatic adjustment of the dosage to the patient to be treated. A medicament typically contains from about 0.01 mg to about 500 mg of the active ingredient, preferably, from about 0.01 mg to about 100 3000 mg of active ingredient.

An effective amount of the drug is ordinarily supplied at a dosage level of from about 0.0002 mg/kg to about 20 mg/kg of body weight per day. Preferably, the range is from about 0.001 to 10 mg/kg of body weight per day, and especially from about 0.001 mg/kg to 7 mg/kg of body weight per day. The compounds may be administered on a regimen of 1 to 4 times per day.

Compounds of the present invention may be used alone at appropriate dosages defined by routine testing in order to obtain optimal antagonism of the human α₁ₐ/α_{1d} adrenergic receptor while minimizing any potential toxicity. In addition, co-administration or sequential administration of other agents that alleviate the effects of BPH is desirable.

Thus, in one embodiment, the method of the present invention includes administration of compounds of this invention and a human testosterone 5-α reductase inhibitor, including inhibitors of 5-α reductase isoenzyme-2.

The dosages of the α₁ₐ adrenergic receptor and testosterone 5-α reductase inhibitors are adjusted when combined to achieve desired effects. As those skilled in the art will appreciate, dosages of the 5-α reductase inhibitor and the α₁ₐ adrenergic receptor antagonist may be independently optimized and combined to achieve a synergistic result wherein the pathology is reduced more than it would be if either agent were used alone. In accordance with the method of the present invention, the individual components of the combination can be administered separately at different times during the course of therapy or concurrently in divided or single combination forms. The instant invention is therefore to be understood as embracing all such regimes of simultaneous or alternating treatment and the term “administering” is to be interpreted accordingly.

Thus, in one embodiment of the present invention, a method of treating BPH is provided which comprises administering to a subject in need of treatment any of the compounds of the present invention in combination with finasteride effective to treat BPH. The dosage of finasteride administered to the subject is about 0.01 mg per subject per day to about 50 mg per subject per day in combination with a α₁ₐ antagonist. Preferably, the dosage of finasteride in the combination is about 0.2 mg per subject per day to about 10 mg per subject per day, more preferably, about 1 to about 7 mg per subject to day, most preferably, about 5 mg per subject per day.

For the treatment of benign prostatic hyperplasia, compounds of this invention exhibiting α₁ₐ adrenergic receptor blockade can be combined with a therapeutically effective amount of a 5α-reductase isoenzyme-2 inhibitor, such as finasteride.

In other embodiments of the present inventions, a method of treating BPH is provided which comprises administering to a subject in need of treatment any of the compounds of the present invention in combination with a therapeutically effective amount of an anti-androgenic agent, androgen receptor antagonists, selective androgen receptor modulators, urinary incontinence drugs (e.g. anti-muscarinics) or 5HT-receptor modulators.

In another embodiment of the present invention, a method of treating BPH is provided which comprises administering to a subject in need of treatment any of the compounds of the present invention in combination with a therapeutically effective amount of a PDE modulator.

### Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

The terms used in describing the invention are commonly used and known to those skilled in the art.

When used herein, the following abbreviations or formulas have the indicated meanings:

| Abbreviation | Meaning |
|---|---|
| Cpd | compound |
| DMF | N,N-dimethyl formamide |
| Et | ethyl |
| EtOAc | ethylacetate |
| HBTU | 2-(1H-benzotriazole-1-yl)-1,1,3,3-tetramethyluronium |
| | hexafluorophosphate |
| Hex | hexane |
| Interm | intermediate |
| Me | methyl |
| MeOH | methanol |
| MS | Mass Spectroscopy |
| m/z | mass/charge |
| Ph | phenyl |
| PhNCO | phenyl isocyanate |
| RT/rt/r.t. | room temperature |
| TEA or Et₃N | triethylamine |
| THP | tetrahydropyran |
| TLC | Thin Layer Chromatography |
| v/v | volume/volume |

Scheme 1 illustrates the preparation of compounds of the present invention representative of a compound of Formula (I).

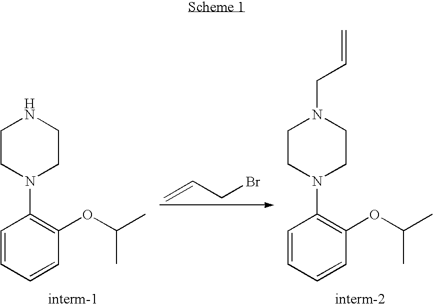

1-(2-isopropoxyphenyl)-piperazine fumarate interm-1 is condensed with allyl bromide and TEA in a solvent such as acetonitrile to provide 1-allyl-4-(2-isopropoxy-phenyl)-piperazine interm-2.

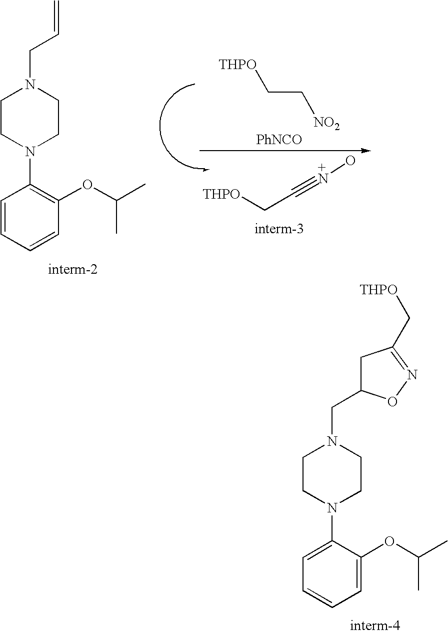

Interm-2 in a solvent such as toluene and the like is reacted with (tetrahydro-pyran-2-yloxy)-acetonitrile N-oxide interm-3 readily generated in situ from 2-(2-nitroethoxy) tetrahydropyran and phenylisocyanate in a solvent such as benzene or toluene and the like in the presence of a base such as TEA and the like to provide 1-(2-isopropoxy-phenyl)-4-[3-(tetrahydro-pyran-2-yloxymethyl)-4,5-dihydro-isoxazol-5-ylmethyl]-piperazine interm-4.

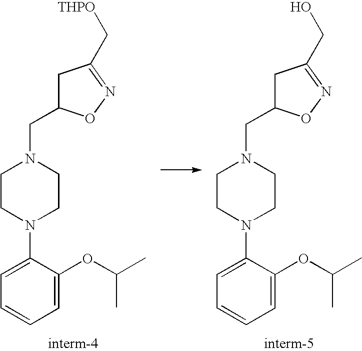

Interm-4 is treated with 1N HCl to provide {5-[4-(2-isopropoxy-phenyl)-piperazin-1-ylmethyl]-4,5-dihydro-isoxazol-3-yl}-methanol interm-5.

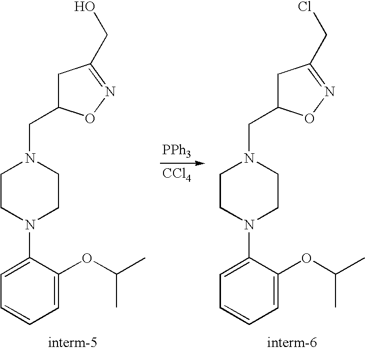

Interm-5 is reacted with triphenylphosphine and a reagent such as carbon tetrachloride and the like to provide 1-(3-chloromethyl-4,5-dihydro-isoxazol-5-ylmethyl)-4-(2-isopropoxy-phenyl)-piperazine interm-6.

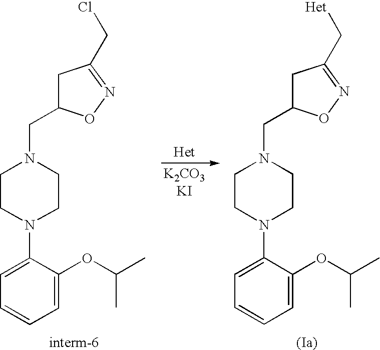

Interm-6 is reacted with Het (as defined previously) in a mixture with one or more reagents such as potassium carbonate and potassium iodide in a solvent such as DMF and the like to provide a compound of Formula (Ia), representative of a compound of Formula (I).

The following examples are offered by way of illustrating Scheme 1; the invention should not be construed as being limited by the chemical reactions and conditions expressed.

### EXAMPLE 1

### 1-{5-[4-(2-isopropoxy-phenyl)-piperazin-1-ylmethyl]-4,5-dihydro-isoxazol-3-ylmethyl}-2,3-dihydro-1H-indole (Compound 4)

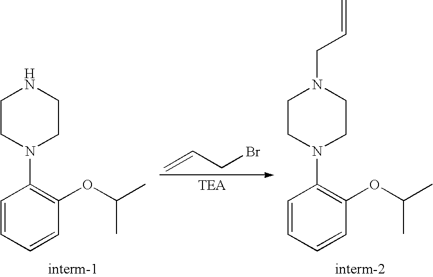

A mixture of 1-(2-isopropoxyphenyl)-piperazine fumarate interm-1 (12 g, 0.036 mol), allyl bromide (5.6 g, 0.046 mol) and triethylamine (9.3 g, 0.092 mol) in acetonitrile (96 mL) was refluxed for 24 h, cooled and concentrated to a syrup. This syrup was treated with 1 N HCl and extracted (2×) into ether. The aqueous layer was neutralized with sodium carbonate, extracted (2×) into chloroform, dried (K₂CO₃), and was concentrated. The resulting syrup was purified via column chromatography (60:30:5 CHCl₃:Hex:NH₃, 2 mol solution in MeOH, v/v) to give 1-allyl-4-(2-isopropoxy-phenyl)-piperazine interm-2 as a light brown syrup (4.74 g, 51%). MS, m/z 261 (M+H).

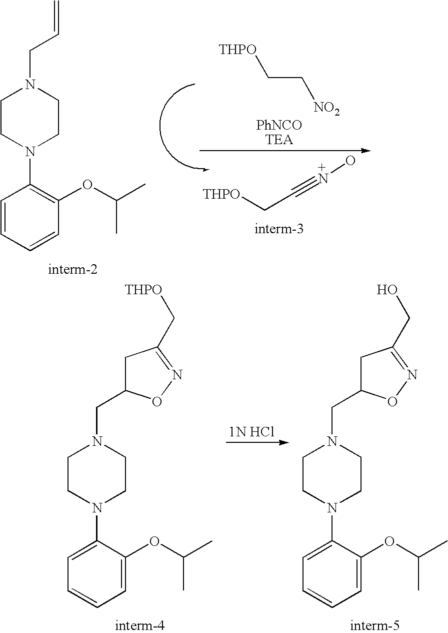

To a solution of interm-2 (2 g, 0.008 mol) in toluene (10 mL) was added 2-(2-nitroethoxy) tetrahydropyran (2.05 g, 0.012 mol), phenylisocyanate (3.66 g, 0.03 mol) and triethylamine (0.1 mL) to form (tetrahydro-pyran-2-yloxy)-acetonitrile N-oxide interm-3 in situ for reaction with interm-2. The mixture was stirred at 30° C. for 18 h, and at 55° C. for another 48 h, then cooled and filtered. The filtrate was treated with water (0.6 mL), stirred for 60 min, then filtered, dried (Na₂SO₄) and concentrated to a dark brown syrup. The syrup was treated with anhydrous ether and hydrolyzed by washing (2×) with 1N HCl to give 1-(2-isopropoxy-phenyl)-4-[3-(tetrahydro-pyran-2-yloxymethyl)-4,5-dihydro-isoxazol-5-ylmethyl]-piperazine interm-4 as an unisolated intermediate. The aqueous layer was made basic with Na₂CO₃ and extracted into chloroform (2×), then dried (Na₂SO₄) and concentrated to a thick brown syrup, which was purified via column chromatography (95:5, CHCl₃:MeOH, v/v) to give {5-[4-(2-isopropoxy-phenyl)-piperazin-1-ylmethyl]-4,5-dihydro-isoxazol-3-yl}-methanol interm-5 (1.02 g, 38%) as a light brown solid. MS, m/z 334 (M+H).

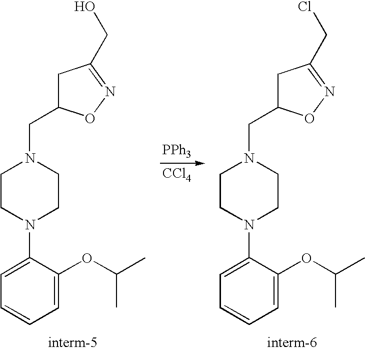

A mixture of interm-5 (1.74 g, 0.005 mol), triphenylphosphine (1.74 g, 0.007 mol) and carbon tetrachloride (60 mL) was refluxed for 24 h, then cooled, concentrated, treated with 1N HCl and extracted with ethyl acetate (2×). The aqueous layer was made basic with Na₂CO₃ and extracted into methylene chloride, then dried (Na₂SO₄) and concentrated to give a brown syrup, which was purified via column chromatography (95:5, CHCl₃: MeOH) to give pure {5-[4-(2-isopropoxy-phenyl)-piperazin-1-ylmethyl]-4,5-dihydro-isoxazol-3-yl}-methanol interm-6 (0.98 g, 56%) as a brown syrup. MS, m/z 352 (M⁺); 354 (M+2).

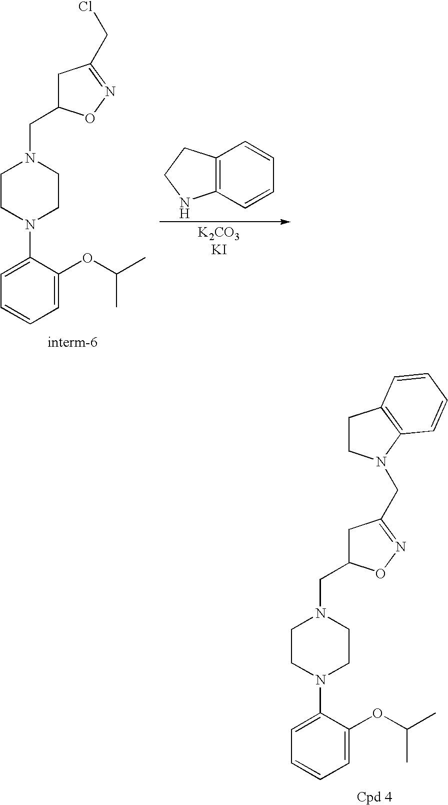

A mixture of interm-6 (0.18 g. 0.5 mmol), indoline (0.07 g, 0.58 mmol), potassium carbonate (0.08 g, 0.58 mmol), and potassium iodide (0.008 g, 0.05 mmol) in DMF (4 mL) was stirred at 98° C. for 24 h and cooled. The reaction mixture was treated with water, extracted into ethyl acetate (2×), then dried (Na₂SO₄) and concentrated to give a syrup, which was purified via preparative TLC (60:30:5 CHCl₃:Hex:NH₃, 2 mol solution in MeOH, v/v) to give pure Compound 4 (0.098 g, 45%) as a pale brown syrup. MS m/z 435 (M+H).

Where the product was obtained as a racemate, the individual enantiomers may be separated using standard techniques, such as chiral column chromatography. For example, chiral column separation of Compound 1 provided Compound 2 and Compound 3.

Following the procedure of Example 1 and substituting the appropriate starting materials, reagents, and solvents the following compounds were prepared (MS represents MS m/z (M+H)^{a}, (M+2)^{b} or (calculated)^{c}):

| Cpd | Name | MS |
|---|---|---|
| 1 | 1-{5-[4-(2-isopropoxy-phenyl)-piperazin-1-ylmethyl]-4,5- | 415^{c} |
| | dihydro-isoxazol-3-ylmethyl}-piperidin-2-one | |
| 2 | 1-{(5R*)-5-[4-(2-isopropoxy-phenyl)-piperazin-1- | 415^{c} |
| | ylmethyl]-4,5-dihydro-isoxazol-3-ylmethyl}- | |
| | piperidin-2-one | |
| 3 | 1-{(5S*)-5-[4-(2-isopropoxy-phenyl)-piperazin-1- | 415^{c} |
| | ylmethyl]-4,5-dihydro-isoxazol-3-ylmethyl}- | |
| | piperidin-2-one | |
| 4a | 1-{5-[4-(2-isopropoxy-phenyl)-piperazin-1-ylmethyl]-4,5- | 480^{a} |
| | dihydro-isoxazol-3-ylmethyl}-6-nitro-2,3-dihydro- | |
| | 1H-indole | |
| 5 | 1-(2-isopropoxy-phenyl)-4-(3-piperidin-1-ylmethyl-4,5- | 401^{a} |
| | dihydro-isoxazol-5-ylmethyl)-piperazine | |

### EXAMPLE 2

### 1-{5-[4-(2-isopropoxy-phenyl)-piperazin-1-ylmethyl]-4,5-dihydro-isoxazol-3-ylmethyl}-2,3-dihydro-1H-indol-6-ylamine (Compound 6)

### (2,6-difluoro-benzyl)-(1-{5-[4-(2-isopropoxy-phenyl)-piperazin-1-ylmethyl]-4,5-dihydro-isoxazol-3-ylmethyl}-2,3-dihydro-1H-indol-6-yl)-amine (Compound 7)

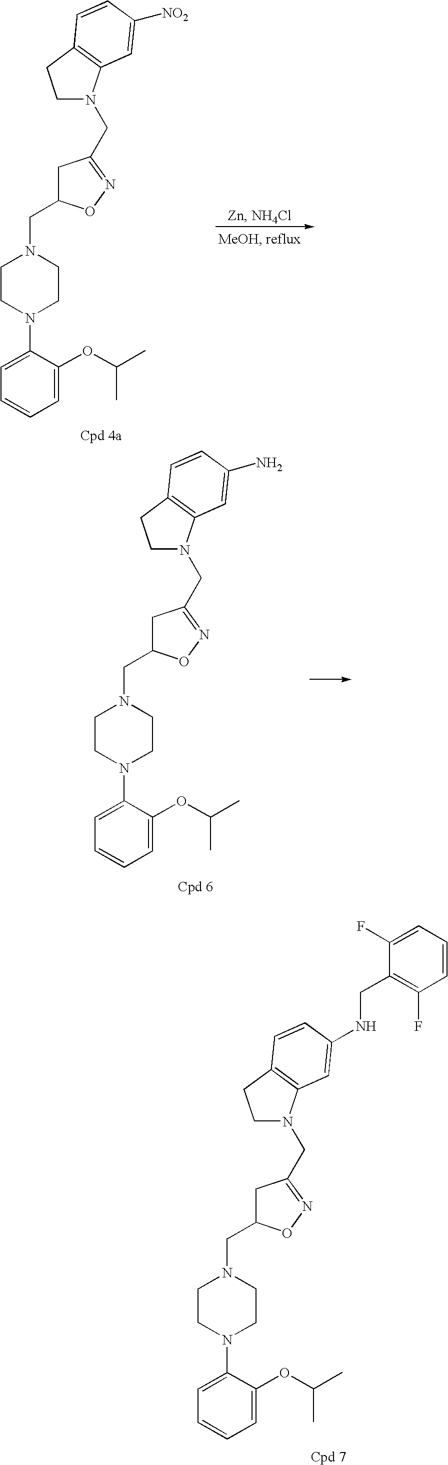

To a solution of Compound 4a (0.7 g, 1.4 mmol) in methanol (30 ml), was added zinc (2.3 g, 35 mmol) and ammonium chloride (0.39 g, 7.2 mmol) and the mixture was refluxed for 30 min and cooled, filtered and concentrated. The resultant syrup was redissolved in chloroform, washed with 10% Na₂CO₃, brine, dried (Na₂SO₄) and concentrated to give Compound 6 (0.53 g, 89%) as a light brown syrup. MS, m/z 450 (M+H).

A solution of Compound 6 (0.11 g, 0.24 mmol) in dichloromethane (5 ml) was treated with 2,6-difluorobenzaldehyde (0.035 g, 0.24 mmol), glacial acetic acid (0.02 ml), and sodium triacetoxyborohydride (0.12 g, 0.56 mmol) and was stirred under an inert atmosphere for 24 h. The reaction mixture was treated with 1N NaOH (8 ml) and stirred for 60 min, and the layers separated. The organic layer was dried (Na₂SO₄), filtered, and concentrated in vacuo to a solid, which was purified via preparative TLC (40:20:2, EtOAc: Hex: MeOH, v/v) to give pure Compound 7 (17 mg, 12.3%) as a brown solid. MS, m/z 576 (M+H).

Following the procedure of Example 2 and substituting the appropriate starting materials, reagents, and solvents the following compounds were prepared (MS represents MS m/z (M+H)):

| Cpd | Name | MS |
|---|---|---|
| 8 | (2-fluoro-benzyl)-(1-{5-[4-(2-isopropoxy-phenyl)-piperazin- | 558 |
| | 1-ylmethyl]-4,5-dihydro-isoxazol-3-ylmethyl}- | |
| | 2,3-dihydro-1H-indol-6-yl)-amine | |
| 9 | (1-{5-[4-(2-isopropoxy-phenyl)-piperazin-1-ylmethyl]-4,5- | 570 |
| | dihydro-isoxazol-3-ylmethyl}-2,3-dihydro-1H-indol-6- | |
| | yl)-(3-methoxy-benzyl)-amine | |

Scheme 2 illustrates the preparation of a compound of Formula (Ib), representative of a compound of Formula (I).

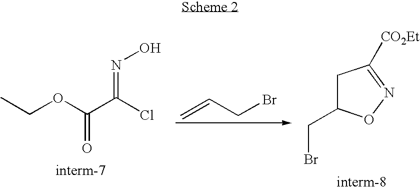

2-chloro-2-hydroxyiminoacetic acid ethyl ester interm-7 is reacted with allyl bromide in a solvent such as diethyl ether to provide 5-bromomethyl-4,5-dihydro-isoxazole-3-carboxylic acid ethyl ester interm-8.

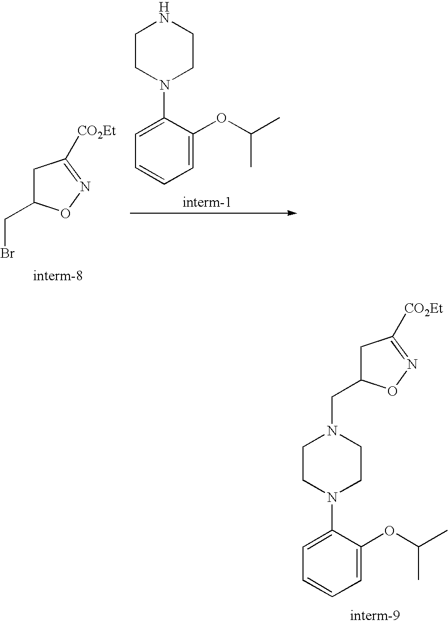

Interm-8 is reacted with 1-(2-isopropoxy-phenyl)-piperazine Interm-1 in a mixture with one or more reagents such as potassium carbonate and potassium iodide in a solvent such as acetonitrile and the like to provide 5-[4-(2-isopropoxy-phenyl)-piperazin-1-ylmethyl]-4,5-dihydro-isoxazole-3-carboxylic acid ethyl ester interm-9.

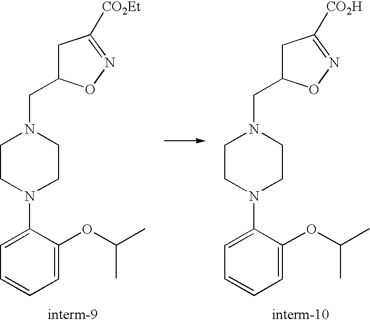

A solution of interm-9 in a solvent such as methanol and the like is reacted in the presence of a base such as NaOH and the like to provide 5-[4-(2-isopropoxy-phenyl)-piperazin-1-ylmethyl]-4,5-dihydro-isoxazole-3-carboxylic acid interm-10.

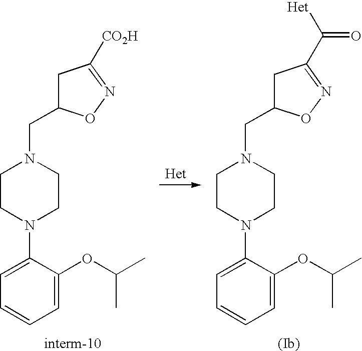

Interm-10 is reacted with Het (as defined previously) in a mixture with one or more reagents such as HBTU and DIPEA in a solvent such as DMF and the like to provide a compound of Formula (Ib), representative of a compound of Formula (I).

The following examples are offered by way of illustration of Scheme 2; the invention should not be construed as being limited by the chemical reactions and conditions expressed.

### EXAMPLE 3

### (2,3-dihydro-indol-1-yl)-{5-[4-(2-isopropoxy-phenyl)-piperazin-1-ylmethyl]-4,5-dihydro-isoxazol-3-yl}-methanone (Compound 10)

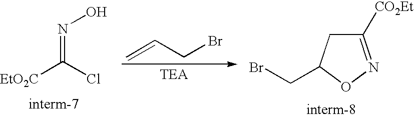

To a cold solution of allyl bromide (26.8 g, 0.221 mol) in diethyl ether (90 ml), was added dropwise a solution of 2-chloro-2-hydroxyiminoacetic acid ethyl ester interm-7 (22.7 g, 0.15 mol) in toluene (60 ml), and mixture stirred for 10 min (0° C.). A mixture of triethylamine (16.69 g, 0.165 mol) in diethyl ether (68 ml) was added. The resulting mixture was stirred at 0° C. for 60 min, and at room temperature for an additional 90 min. The solution was filtered and the filtrate was washed with 1N HCl and brine, then dried (Na₂SO₄) and concentrated to a syrup which was purified via column chromatography (4:1;Hex:EtOAc, v/v) to give pure 5-bromomethyl-4,5-dihydro-isoxazole-3-carboxylic acid ethyl ester interm-8 (15.3 g, 43%) as a colorless crystalline solid. MS, m/z 236 (M⁺), 238 (M+2).

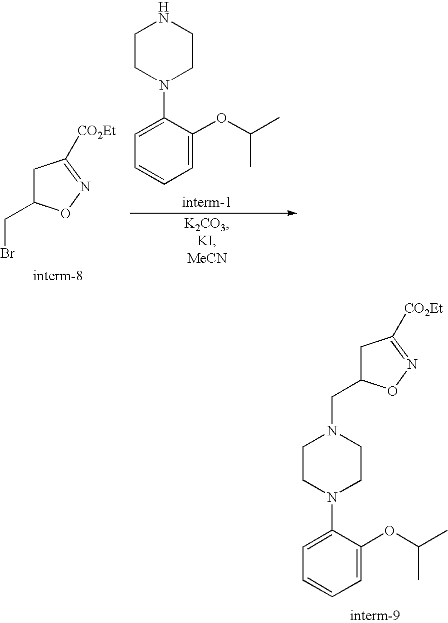

A mixture of interm-8 (6.0 g, 25 mmol), 1-(2-isopropoxyphenyl)-piperazine fumarate interm-1 (8.5 g, 25 mmol), potassium carbonate (10.5 g, 76 mmol), and potassium iodide (1.05 g, 6.3 mmol) in acetonitrile (100 ml) was refluxed for 24 hr, then cooled, filtered and concentrated. The resulting syrup was redissolved into CH₂Cl₂, and washed with water, then dried (Na₂SO₄) and concentrated to a syrup, which was purified via column chromatography (40:20:1; Hexane: EtOAc: MeOH, v/v) to give 5-[4-(2-isopropoxy-phenyl)-piperazin-1-ylmethyl]-4,5-dihydro-isoxazole-3-carboxylic acid ethyl ester interm-9 (5.8 g, 61%) as a colorless syrup. MS, m/z 376 (M+H).

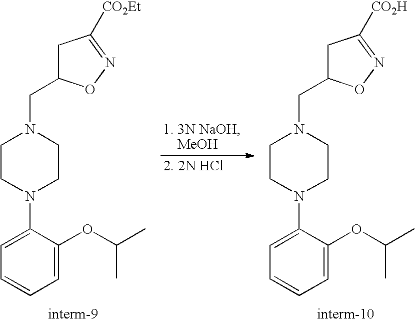

A mixture of interm-9 (2.4 g, 6.4 mmol), in methanol (24 ml) and 3N NaOH (48 ml) was heated at 80° C. for 24 hr. The reaction mixture was cooled, and treated with 1N HCl (pH=6-7), then filtered and concentrated to provide 5-[4-(2-isopropoxy-phenyl)-piperazin-1-ylmethyl]-4,5-dihydro-isoxazole-3-carboxylic acid interm-10 (2.2 g, 99%) as an off white solid. MS, m/z 348 (M+H).

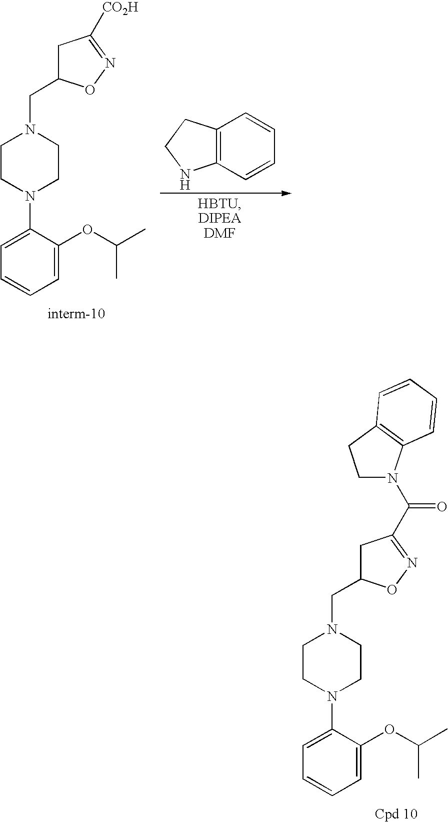

To a solution of interm-10 (0.38 g, 1.0 mol) in DMF (6 mol) was added indoline (0.13 g, 1.0 mol), HBTU (0.42 g, 1.1 mol) and diisopropylethyl amine (DIPEA) (0.1 μg, 1.6 mmol). The mixture was stirred for 24 hr at room temperature, treated with water and extracted into EtOAc, then dried (Na₂SO₄) and concentrated to a syrup which was purified via column chromatography (40:20:1; EtOAc:Hex:MeOH, v/v) to give pure Compound 10 (0.1 g, 24%) as a syrup. MS, m/z 449 (M+H).

Where the product was obtained as a racemate, the individual enantiomers may be separated using standard techniques, such as chiral column chromatography. For example, chiral column separation of Compound 10 provided Compound 36 and Compound 37; Compound 18 provided Compound 40 and Compound 41; Compound 19 provided Compound 42 and Compound 43; Compound 28 provided Compound 44 and Compound 45; and, Compound 29 provided Compound 38 and Compound 39.

Following the procedure of Example 3 and substituting the appropriate starting materials, reagents, and solvents the following compounds were prepared (MS represents MS m/z (M+H)^{a}, (M+2)_{b} or (calculated)_{c}; m.p. shown in ° C.):

| Cpd | Name | MS |
|---|---|---|
| 11 | {5-[4-(2-isopropoxy-phenyl)-piperazin-1-ylmethyl]-4,5-dihydro- | 494^{c} |
| | isoxazol-3-yl}-(6-nitro-2,3-dihydro-indol-1-yl)-methanone | |
| 12 | {5-[4-(2-isopropoxy-phenyl)-piperazin-1-ylmethyl]-4,5-dihydro- | 415^{a} |
| | isoxazol-3-yl}-piperidin-1-yl-methanone | |
| 13 | (5-bromo-2,3-dihydro-indol-1-yl)-{5-[4-(2-isopropoxy-phenyl)- | 529^{b} |
| | piperazin-1-ylmethyl]-4,5-dihydro-isoxazol-3-yl}-methanone | |
| 16 | (5-chloro-2,3-dihydro-indol-1-yl)-{5-[4-(2-isopropoxy-phenyl)- | 483^{a} |
| | piperazin-1-ylmethyl]-4,5-dihydro-isoxazol-3-yl}-methanone | |
| 17 | [4-(2-isopropoxy-phenyl)-piperazin-1-yl]-{5-[4-(2-isopropoxy- | 550^{a} |
| | phenyl)-piperazin-1-ylmethyl]-4,5-dihydro-isoxazol-3-yl}-methanone | |
| 18 | [4-(4-fluoro-benzoyl)-piperidin-1-yl]-{5-[4-(2-isopropoxy-phenyl)- | 537^{a} |
| | piperazin-1-ylmethyl]-4,5-dihydro-isoxazol-3-yl}-methanone | |
| 19 | (5-bromo-3,3-dimethyl-2,3-dihydro-indol-1-yl)-{5-[4-(2-isopropoxy- | 555^{a}, |
| | phenyl)-piperazin-1-ylmethyl]-4,5-dihydro-isoxazol-3-yl}-methanone | 557^{a} |
| 20 | (5-fluoro-2,3-dihydro-indol-1-yl)-{5-[4-(2-isopropoxy-phenyl)- | 467^{a} |
| | piperazin-1-ylmethyl]-4,5-dihydro-isoxazol-3-yl}-methanone | |
| 21 | (4-benzoyl-piperidin-1-yl)-{5-[4-(2-isopropoxy-phenyl)-piperazin-1- | 519^{a} |
| | ylmethyl]-4,5-dihydro-isoxazol-3-yl}-methanone | |
| 22 | 5-[4-(2-isopropoxy-phenyl)-piperazin-1-ylmethyl]-4,5-dihydro- | 506^{a} |
| | isoxazole-3-carboxylic acid (1-acetyl-2,3-dihydro-1H-indol-5-yl)-amide | |
| 23 | (5,6-dimethoxy-2,3-dihydro-indol-1-yl)-{5-[4-(2-isopropoxy-phenyl)- | 509^{a} |
| | piperazin-1-ylmethyl]-4,5-dihydro-isoxazol-3-yl}-methanone | |
| 24 | 5-[4-(2-isopropoxy-phenyl)-piperazin-1-ylmethyl]-4,5-dihydro- | 506^{a} |
| | isoxazole-3-carboxylic acid (1-acetyl-2,3-dihydro-1H-indol-6-yl)-amide | |
| 25 | {5-[4-(2-isopropoxy-phenyl)-piperazin-1-ylmethyl]-4,5-dihydro- | 463^{a} |
| | isoxazol-3-yl}-(5-methyl-2,3-dihydro-indol-1-yl)-methanone | |
| 26 | (3,4-dihydro-2H-quinolin-1-yl)-{5-[4-(2-isopropoxy-phenyl)- | 463^{a} |
| | piperazin-1-ylmethyl]-4,5-dihydro-isoxazol-3-yl}-methanone | |
| 27 | (4-benzyl-piperidin-1-yl)-{5-[4-(2-isopropoxy-phenyl)-piperazin-1- | 505^{a} |
| | ylmethyl]-4,5-dihydro-isoxazol-3-yl}-methanone | |
| 28 | {5-[4-(2-isopropoxy-phenyl)-piperazin-1-ylmethyl]-4,5-dihydro- | 417^{a} |
| | isoxazol-3-yl}-morpholin-4-yl-methanone | |
| 29 | {5-[4-(2-isopropoxy-phenyl)-piperazin-1-ylmethyl]-4,5-dihydro- | 507^{a} |
| | isoxazol-3-yl}-(4-phenoxy-piperidin-1-yl)-methanone | |
| 30 | (4-benzenesulfonyl-piperidin-1-yl)-{5-[4-(2-isopropoxy-phenyl)- | 555^{a} |
| | piperazin-1-ylmethyl]-4,5-dihydro-isoxazol-3-yl}-methanone, m.p. 74-76° C. | |
| 31 | (4,4-diphenyl-piperidin-1-yl)-{5-[4-(2-isopropoxy-phenyl)-piperazin- | 567^{a} |
| | 1-ylmethyl]-4,5-dihydro-isoxazol-3-yl}-methanone, m.p. 61-63° C. | |
| 32 | {5-[4-(2-isopropoxy-phenyl)-piperazin-1-ylmethyl]-4,5-dihydro- | 508^{a} |
| | isoxazol-3-yl}-[4-(pyridin-2-yloxy)-piperidin-1-yl]-methanone | |
| 33 | {5-[4-(2-isopropoxy-phenyl)-piperazin-1-ylmethyl]-4,5-dihydro- | 621^{a} |
| | isoxazol-3-yl}-[4-(2-methoxy-phenyl)-piperidin-1-yl]-methanone | |
| 34 | [4-(4-chloro-phenoxy)-piperidin-1-yl]-{5-[4-(2-isopropoxy-phenyl)- | 541^{a} |
| | piperazin-1-ylmethyl]-4,5-dihydro-isoxazol-3-yl}-methanone | |
| 35 | {5-[4-(2-isopropoxy-phenyl)-piperazin-1-ylmethyl]-4,5-dihydro- | 401^{a} |
| | isoxazol-3-yl}-pyrrolidin-1-yl-methanone | |
| 36 | (2,3-dihydro-indol-1-yl)-{(5S*)-5-[4-(2-isopropoxy-phenyl)- | 449^{c} |
| | piperazin-1-ylmethyl]-4,5-dihydro-isoxazol-3-yl}-methanone | |
| 37 | (2,3-dihydro-indol-1-yl)-{(5R*)-5-[4-(2-isopropoxy-phenyl)- | 449^{c} |
| | piperazin-1-ylmethyl]-4,5-dihydro-isoxazol-3-yl}-methanone | |
| 38 | {(5R*)-5-[4-(2-isopropoxy-phenyl)-piperazin-1-ylmethyl]-4,5- | 507^{c} |
| | dihydro-isoxazol-3-yl}-(4-phenoxy-piperidin-1-yl)-methanone | |
| 39 | {(5S*)-5-[4-(2-isopropoxy-phenyl)-piperazin-1-ylmethyl]-4,5- | 507^{c} |
| | dihydro-isoxazol-3-yl}-(4-phenoxy-piperidin-1-yl)-methanone | |
| 40 | [4-(4-fluoro-benzoyl)-piperidin-1-yl]-{(5S*)-5-[4-(2-isopropoxy- | 537^{c} |
| | phenyl)-piperazin-1-ylmethyl]-4,5-dihydro-isoxazol-3-yl}-methanone | |
| 41 | [4-(4-fluoro-benzoyl)-piperidin-1-yl]-{(5R*)-5-[4-(2-isopropoxy- | 537^{c} |
| | phenyl)-piperazin-1-ylmethyl]-4,5-dihydro-isoxazol-3-yl}-methanone | |
| 42 | (5-bromo-3,3-dimethyl-2,3-dihydro-indol-1-yl)-{(5R*)-5-[4-(2- | 556^{c} |
| | isopropoxy-phenyl)-piperazin-1-ylmethyl]-4,5-dihydro-isoxazol-3- | |
| | yl}-methanone | |
| 43 | (5-bromo-3,3-dimethyl-2,3-dihydro-indol-1-yl)-{(5S*)-5-[4-(2- | 556^{c} |
| | isopropoxy-phenyl)-piperazin-1-ylmethyl]-4,5-dihydro-isoxazol-3-yl}-methanone | |
| 44 | {(5S*)-5-[4-(2-isopropoxy-phenyl)-piperazin-1-ylmethyl]-4,5- | 417^{c} |
| | dihydro-isoxazol-3-yl}-morpholin-4-yl-methanone | |
| 45 | {(5R*)-5-[4-(2-Isopropoxy-phenyl)-piperazin-1-ylmethyl]-4,5- | 417^{c} |
| | dihydro-isoxazol-3-yl}-morpholin-4-yl-methanone | |

### EXAMPLE 4

### (6-amino-2,3-dihydro-indol-1-yl)-{5-[4-(2-isopropoxy-phenyl)-piperazin-1-ylmethyl]-4,5-dihydro-isoxazol-3-yl}-methanone (Compound 14)

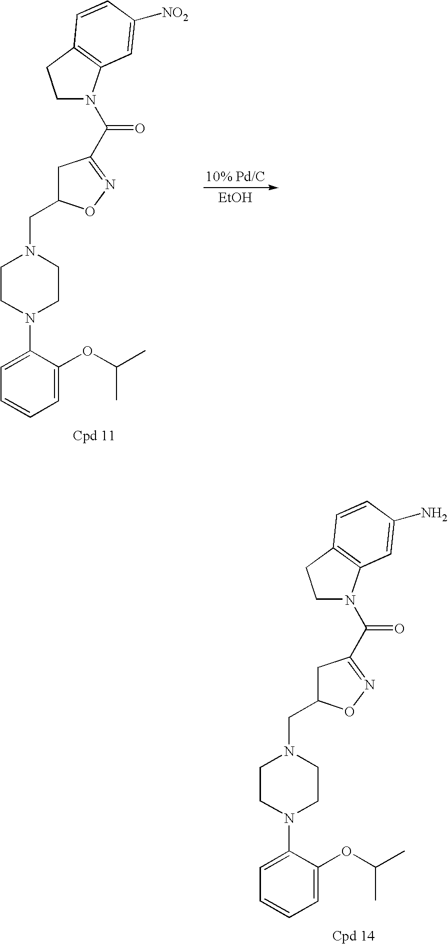

A solution of Compound 11 (0.15 g, 0.32 mmol) in THF/EtOH (7 mL/15 mL) was treated with 10% Pd/C (0.06 g) under hydrogen (40 psi) for 90 min, then filtered and concentrated to a syrup which was purified via column chromatography (40:20:3 EtOAc:Hex:MeOH, v/v) to give Compound 14 (52 mg, 37%) as a light yellow solid. MS, m/z 464 (M+H).

### EXAMPLE 5

### [6-(2-fluoro-benzylamino)-2,3-dihydro-indol-1-yl]-{5-[4-(2-isopropoxy-phenyl)-piperazin-1-ylmethyl]-4,5-dihydro-isoxazol-3-yl}-methanone (Compound 15)

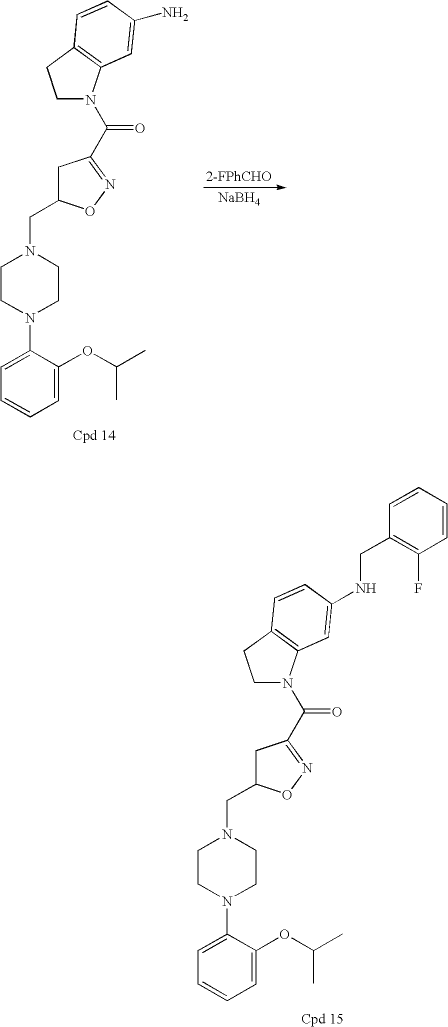

A solution of Compound 14 (43 mg, 0.09 mmol) in methanol (4 ml) was treated with 2-fluorobenzaldehyde (12 mg, 0.09 mmol) and stirred for 2.5 hr, and sodium borohydride (6 mg, 0.16 mmol) was added. The mixture was stirred for an additional 1.5 hr and concentrated. The resultant syrup was dissolved in CH₂Cl₂, washed with 1N NaOH, then dried (Na₂SO₄) and concentrated to provide Compound 15 (36 mg, 70%) as a yellow syrup. MS, m/z 572 (M+H).

### BIOLOGICAL EXAMPLES

### α1—Adrenergic Receptor Binding Assay

### Preparation of COS Cell Membranes

Membranes were prepared from COS-7 cells (African Green monkey kidney SV40-transformed cells) that had been transfected with one of the three α₁-AR subtypes by the following method: COS cells from ten 100 mm tissue culture plates were scraped into a 5 ml volume of TE (a mixture of 50 mM tris(hydroxymethyl)aminomethane hydrochloride (Tris-HCl) and 5 mM ethylenediaminetetraacetic acid (EDTA) at pH 7.4). The cell suspension was disrupted with a Brinkman Polytron (at a setting of 8) for 10 sec. The disrupted cells were centrifuged at 1000×g for 10 min at 4° C. Supernatants were centrifuged at 34,500×g for 20 min at 4° C. The membrane pellets were suspended in a 2 ml volume of TNE (a mixture of 50 mM Tris-HCl, 5 mM EDTA and 150 mM NaCl at pH7.4). An aliquot of the membrane suspension was stored at −70° C. until use. The protein concentration was determined using a BioRad “DC” protein assay kit following membrane solubilization with Triton X-100.

### Radio-ligand Binding Assay

Triplicate determinations of radio-ligand binding in the presence of increasing concentrations of testing compound were made. The reagents were added to 96-well polypropylene plate wells. Each assay well contained 140 μL TNE, 25 μL 125′-2-(β-4-hydroxyphenyl)ethylaminomethyltetralone (¹²⁵I-HEAT) (specific activity 2200 Ci/mmol, Dupont-New England Nuclear, 50 μM final), 10 μL testing compound dissolved in dimethyl sulfoxide (DMSO) (1 μM to 10 μM in half-log increments, final), and 25 μL appropriate α₁-AR membrane subtype suspension in TNE (0.5 ng/μL for the α₁ₐ and α_{1b} subtypes and 13 ng/μL for the α_{1d} subtype). The plate was incubated at rt for 1 hr. The contents of the wells were filtered through a glass filter (type C) (GF/C) membrane Unifilter plate (Packard Instruments) using the Packard Filtermate cell harvester. The filter plates were dried in a vacuum oven for 30 min at 40° C. 25 μL Microscint 20 liquid scintillation fluid (Packard Instuments) was added to each well. The radioactive content was analyzed in the TopCount microplate scintillation counter (Packard Instruments).

### Data Analysis

The Kᵢ values (in nM) shown in Table 1 were determined using GraphPad Prism software. K_{d} values used in the Kᵢ calculation for the α₁-AR subtypes for ¹²⁵I-HEAT were 81.5 nM for the α₁ₐ-AR, 79 nM for the α_{1b}-AR and 50 nM for the α_{1d}-AR: ⁽¹⁾ Average Value, ⁽²⁾ Individual Values.

TABLE
Alpha 1 Ki (nM)

| Cpd | α₁ₐ⁽¹⁾ | α₁ₐ⁽²⁾ | α_{1b}⁽¹⁾ | α_{1b}⁽²⁾ | α_{1d}⁽¹⁾ | α_{1d}⁽²⁾ |
|---|---|---|---|---|---|---|
| 1 | 0.96 | 0.96 | 82 | 82 | 8.5 | 8.5 |
| 2 | 0.79 | 1, 0.7, 0.86, | 53.5 | 79, 41, 56, 38 | 4.5 | 5.2, 4.7, 5.8, 2.3 |
| | | 0.6 | | | | |
| 3 | 2.5 | 2.5 | 215 | 215 | 20 | 20 |
| 4 | 1.9 | 1.9 | 101 | 101 | 10 | 10 |
| 5 | 10 | 10 | 5000 | 5000 | 178 | 178 |
| 6 | 4.3 | 4.3 | 410 | 410 | 46 | 46 |
| 7 | 6.4 | 6.4 | 172 | 172 | 9.4 | 9.4 |
| 8 | 6.9 | 6.9 | 304 | 304 | 35 | 35 |
| 9 | 34 | 34 | 195 | 195 | 22 | 22 |
| 10 | 2.12 | 1.4, 3.4, 3.1, | 107.12 | 90, 145, 92, | 1.302 | 1.1, 2.2, 0.21, |
| | | 1.9, 0.8 | | 87.6, 121 | | 0.5, 2.5 |
| 11 | 1.415 | 2.2, 0.63 | 144.65 | 153, 136.3 | 1.825 | 3.1, 0.55 |
| 12 | 1.806 | 1.018, 1.7, 2.7 | 249 | 334, 174, 239 | 17.753 | 29, 18, 6.259 |
| 13 | 2.477 | 1.308, 2.4, | 191.46 | 402, 147, 134, | 6.081575 | 0.11, 0.3263, |
| | | 3.7, 2.5 | | 82.84 | | 23, 0.89 |
| 14 | 2 | 2 | 141 | 141 | 2.8 | 2.8 |
| 15 | 9.6 | 9.6 | 128 | 128 | 11.6 | 11.6 |
| 16 | 8.3 | 8.3 | 133 | 133 | 4.7 | 4.7 |
| 17 | 16.3 | 16.3 | 187 | 187 | 6.6 | 6.6 |
| 18 | 7.9 | 9.9, 5.9 | 423 | 423, 423 | 21.5 | 22, 21 |
| 19 | 13 | 13, 13 | 280 | 302, 258 | 0.6 | 0.4, 0.8 |
| 20 | 2.225 | 1.35, 3.1 | 111.6 | 97.2, 126 | 0.6225 | 0.145, 1.1 |
| 21 | 5.25 | 3.2, 7.3 | 224 | 288, 160 | 7.65 | 5.5, 9.8 |
| 22 | 2.02 | 2.54, 1.5 | 80.35 | 73, 87.7 | 1.815 | 1.5, 2.13 |
| 23 | 5.65 | 5.65 | 259 | 259 | 6.56 | 6.56 |
| 24 | 5.46 | 5.46 | 101 | 101 | 3.95 | 3.95 |
| 25 | 3.5 | 3.5 | 71 | 71 | 0.12 | 0.12 |
| 26 | 6.7 | 6.7 | 258 | 258 | 11 | 11 |
| 27 | 11.6 | 11.6 | 219 | 219 | 6.5 | 6.5 |
| 28 | 3 | 3 | 691 | 691 | 27.2 | 27.2 |
| 29 | 6.8 | 2.2, 11.4 | 672 | 1011, 333 | 8.2 | 5.6, 10.8 |
| 30 | 3.4 | 3.4, 3.4 | 173.5 | 164, 183 | 4.5 | 5.8, 3.2 |
| 31 | 25 | 25 | 248 | 248 | 9.6 | 9.6 |
| 32 | 41.7 | 41.7 | 264 | 264 | 44.6 | 44.6 |
| 33 | 19 | 19 | 254 | 254 | 20 | 20 |
| 34 | 40 | 40 | 255 | 255 | 27 | 27 |
| 35 | 3.65 | 1.6, 5.7 | 212.5 | 183, 242 | 9 | 11, 7 |
| 36 | 38.18 | 0.36, 76 | 191 | 29, 353 | 90.25 | 0.5, 180 |
| 37 | 0.54 | 0.54 | 37 | 37 | 0.28 | 0.28 |
| 38 | 132 | 132 | 639 | 639 | 55 | 55 |
| 39 | 1.6 | 1.4, 1.8 | 216.5 | 223, 210 | 8.55 | 10.4, 6.7 |
| 40 | 6.436 | 6.436 | 531.2 | 531.2 | 4.606 | 4.606 |
| 41 | 240.4 | 240.4 | 1687 | 1687 | 58.12 | 58.12 |
| 42 | 5.866 | 5.866 | 289.2 | 289.2 | 0.4403 | 0.4403 |
| 43 | 317.1 | 317.1 | 1080 | 1080 | 103.5 | 103.5 |
| 44 | 2.322 | 2.322 | 520.8 | 520.8 | 19.92 | 19.92 |
| 45 | 73.07 | 73.07 | 1061 | 1061 | 297.2 | 297.2 |

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. These publications are hereby incorporated by reference in their entirety into this application to describe more fully the state of the art to which this invention pertains.

What is claimed is:

1. A compound of a Formula (I): 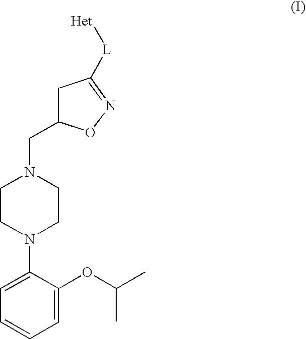
and a form thereof, wherein
L is selected from the group consisting of —CH₂—, —CO— and —CONH—;
Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with C₁₋₈alkyl, amino di-substituted with C₁₋₆alkyl, amino mono-substituted with ArC₁₋₆alkyl, amino disubstituted with ArC₁₋₆alkyl, Ar, Ar—CO, Ar-oxy, and Ar—SO₂;
Ar is selected from pyridinyl, phenyl and phenyl substituted with one or more substituents selected from halo, C₁₋₆alkyl, and C₁₋₆alkyloxy;
with the proviso that 1-[[4,5-dihydro-5-[[4-[2-(1-methylethoxy)phenyl]-1-piperazinyl]methyl]-3-isoxazolyl]methyl]-2-piperidone is not included.

2. The compound of claim 1 and a form thereof wherein Het is selected from the group consisting of pyrrolidinyl, piperidinyl, piperazinyl, 2,3-dihydro-1H-indolyl, 1,2,3,4-tetrahydro-quinolinyl, and morpholinyl.

3. The compound of claim 1 and a form thereof wherein L is —CH₂— or —CO— and wherein Het is connected to L via a nitrogen atom.

4. The compound of claim 1 and a form thereof wherein L is —CONH— and wherein Het is connected to L via a carbon atom.

5. The compound of claim 2 and a form thereof wherein Het is selected from the group consisting of piperidin-2-on-1-yl, piperidin-1-yl, piperidin-1-yl substituted with phenylcarbonyl, 2,3-dihydroindol-1-yl, 2,3-dihydroindol-1-yl substituted with amino, 2,3-dihydroindol-1-yl substituted with difluorophenylmethylamino, 2,3-dihydroindol-1-yl substituted with monofluorophenylmethylamino, 2,3-dihydroindol-1-yl substituted with methoxyphenylmethylamino, 2,3-dihydroindol-1-yl substituted with nitro, 2,3-dihydroindol-1-yl substituted with bromo, 2,3-dihydroindol-1-yl substituted with chloro, piperazin-1-yl substituted with 2-(2-methylethyloxy), 5-bromo-(3,3-dimethyl)-2,3-dihydro-indol-1-yl, 5-fluoro-2,3-dihydro-indol-1-yl, 2,3-dihydro-indol-1-yl substituted with methyl, 2,3-dihydro-indolyl substituted with methylcarbonyl, 2,3-dihydro-indol-1-yl substituted with two methoxy substituents, piperidin-1-yl substituted with 4-chloro-phenyloxy, piperidin-1-yl substituted with 4-fluorophenylcarbonyl, 1,2,3,4-tetrahydro-quinolin-1-yl, piperidin-1-yl substituted with phenylmethyl, morpholin-4-yl, piperidin-1-yl substituted with phenyloxy, piperidin-1-yl substituted with phenyl sulphonyl, piperidin-1-yl substituted with two phenyls, piperidin-1-yl substituted with pyridin-4-yloxy, piperidin-1-yl substituted with phenyl substituted with methoxy, and pyrrolidin-1-yl.

6. The compound of claim 1 and a form thereof wherein Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with ArC₁₋₆alkyl, Ar—CO, Ar-oxy, and Ar—SO₂.

7. The compound of claim 2 and a form thereof wherein Het is selected from the group consisting of pyrrolidinyl, piperidinyl, 2,3-dihydro-1H-indolyl, 1,2,3,4-tetrahydro-quinolinyl, and morpholinyl.

8. The compound of claim 5 and a form thereof wherein Het is selected from the group consisting of piperidin-2-on-1-yl, piperidin-1-yl, piperidin-1-yl substituted with phenylcarbonyl, 2,3-dihydroindol-1-yl, 2,3-dihydroindol-1-yl substituted with amino, 2,3-dihydroindol-1-yl substituted with difluorophenylmethylamino, 2,3-dihydroindol-1-yl substituted with monofluorophenylmethylamino, 2,3-dihydroindol-1-yl substituted with nitro, 2,3-dihydroindol-1-yl substituted with bromo, 2,3-dihydroindol-1-yl substituted with chloro, 5-bromo-(3,3-dimethyl)-2,3-dihydro-indol-1-yl, 5-fluoro-2,3-dihydro-indol-1-yl, 2,3-dihydro-indol-1-yl substituted with methyl, 2,3-dihydro-indolyl substituted with methylcarbonyl, 2,3-dihydro-indol-1-yl substituted with two methoxy substituents, piperidin-1-yl substituted with 4-fluorophenylcarbonyl, 1,2,3,4-tetrahydro-quinolin-1-yl, morpholin-4-yl, piperidin-1-yl substituted with phenyloxy, piperidin-1-yl substituted with phenyl sulphonyl and pyrrolidin-1-yl.

9. The compound of claim 1 selected from the group consisting of:
1-{(5R*)-5-[4-(2-isopropoxy-phenyl)-piperazin-1-ylmethyl]-4,5-dihydro-isoxazol-3-ylmethyl}-piperidin-2-one,
1-{(5S*)-5-[4-(2-isopropoxy-phenyl)-piperazin-1-ylmethyl]-4,5-dihydro-isoxazol-3-ylmethyl}-piperidin-2-one,
1-{5-[4-(2-isopropoxy-phenyl)-piperazin-1-ylmethyl]-4,5-dihydro-isoxazol-3-ylmethyl}-2,3-dihydro-1H-indole,
1-{5-[4-(2-isopropoxy-phenyl)-piperazin-1-ylmethyl]-4,5-dihydro-isoxazol-3-ylmethyl}-6-nitro-2,3-dihydro-1H-indole,
1-(2-isopropoxy-phenyl)-4-(3-piperidin-1-ylmethyl-4,5-dihydro-isoxazol-5-ylmethyl)-piperazine,
1-{5-[4-(2-isopropoxy-phenyl)-piperazin-1-ylmethyl]-4,5-dihydro-isoxazol-3-ylmethyl}-2,3-dihydro-1H-indol-6-ylamine,
(2,6-difluoro-benzyl)-(1-{5-[4-(2-isopropoxy-phenyl)-piperazin-1-ylmethyl]-4,5-dihydro-isoxazol-3-ylmethyl}-2,3-dihydro-1H-indol-6-yl)-amine,
(2-fluoro-benzyl)-(1-{5-[4-(2-isopropoxy-phenyl)-piperazin-1-ylmethyl]-4,5-dihydro-isoxazol-3-ylmethyl}-2,3-dihydro-1H-indol-6-yl)-amine,
(1-{5-[4-(2-isopropoxy-phenyl)-piperazin-1-ylmethyl]-4,5-dihydro-isoxazol-3-ylmethyl}-2,3-dihydro-1H-indol-6-yl)-(3-methoxy-benzyl)-amine,
(2,3-dihydro-indol-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-(6-nitro-2,3-dihydro-indol-1-yl)-methanone,
{5-[4-(2-isopropoxy-phenyl)-piperazin-1-ylmethyl]-4,5-dihydro-isoxazol-3-yl}-piperidin-1-yl-methanone,
(5-bromo-2,3-dihydro-indol-1-yl)-{5-[4-(2-isopropoxy-phenyl)-piperazin-1-ylmethyl]-4,5-dihydro-isoxazol-3-yl}-methanone,
(6-amino-2,3-dihydro-indol-1-yl)-{5-[4-(2-isopropoxy-phenyl)-piperazin-1-ylmethyl]-4,5-dihydro-isoxazol-3-yl}-methanone,
[6-(2-fluoro-benzylamino)-2,3-dihydro-indol-1-yl]-{5-[4-(2-isopropoxy-phenyl)-piperazin-1-ylmethyl]-4,5-dihydro-isoxazol-3-yl}-methanone,
(5-chloro-2,3-dihydro-indol-1-yl)-{5-[4-(2-isopropoxy-phenyl)-piperazin-1-ylmethyl]-4,5-dihydro-isoxazol-3-yl}-methanone,
[4-(2-isopropoxy-phenyl)-piperazin-1-yl]-{5-[4-(2-isopropoxy-phenyl)-piperazin-1-ylmethyl]-4,5-dihydro-isoxazol-3-yl}-methanone,
[4-(4-fluoro-benzoyl)-piperidin-1-yl]-{5-[4-(2-isopropoxy-phenyl)-piperazin-1-ylmethyl]-4,5-dihydro-isoxazol-3-yl}-methanone,
(5-bromo-3,3-dimethyl-2,3-dihydro-indol-1-yl)-{5-[4-(2-isopropoxy-phenyl)-piperazin-1-ylmethyl]-4,5-dihydro-isoxazol-3-yl}-methanone,
(5-fluoro-2,3-dihydro-indol-1-yl)-{5-[4-(2-isopropoxy-phenyl)-piperazin-1-ylmethyl]-4,5-dihydro-isoxazol-3-yl}-methanone,
(4-benzoyl-piperidin-1-yl)-{5-[4-(2-isopropoxy-phenyl)-piperazin-1-ylmethyl]-4,5-dihydro-isoxazol-3-yl}-methanone,
5-[4-(2-isopropoxy-phenyl)-piperazin-1-ylmethyl]-4,5-dihydro-isoxazole-3-carboxylic acid (1-acetyl-2,3-dihydro-1H-indol-5-yl)-amide,
(5,6-dimethoxy-2,3-dihydro-indol-1-yl)-{5-[4-(2-isopropoxy-phenyl)-piperazin-1-ylmethyl]-4,5-dihydro-isoxazol-3-yl}-methanone,
5-[4-(2-isopropoxy-phenyl)-piperazin-1-ylmethyl]-4,5-dihydro-isoxazole-3-carboxylic acid (1-acetyl-2,3-dihydro-1H-indol-6-yl)-amide,
{5-[4-(2-isopropoxy-phenyl)-piperazin-1-ylmethyl]-4,5-dihydro-isoxazol-3-yl}-(5-methyl-2,3-dihydro-indol-1-yl)-methanone,
(3,4-dihydro-2H-quinolin-1-yl)-{5-[4-(2-isopropoxy-phenyl)-piperazin-1-ylmethyl]-4,5-dihydro-isoxazol-3-yl}-methanone,
(4-benzyl-piperidin-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-morpholin-4-yl-methanone,
{5-[4-(2-isopropoxy-phenyl)-piperazin-1-ylmethyl]-4,5-dihydro-isoxazol-3-yl}-(4-phenoxy-piperidin-1-yl)-methanone,
(4-benzenesulfonyl-piperidin-1-yl)-{5-[4-(2-isopropoxy-phenyl)-piperazin-1-ylmethyl]-4,5-dihydro-isoxazol-3-yl}-methanone,
(4,4-diphenyl-piperidin-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-[4-(pyridin-2-yloxy)-piperidin-1-yl]-methanone,
{5-[4-(2-isopropoxy-phenyl)-piperazin-1-ylmethyl]-4,5-dihydro-isoxazol-3-yl}-[4-(2-methoxy-phenyl)-piperidin-1-yl]-methanone,
[4-(4-chloro-phenoxy)-piperidin-1-yl]-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-pyrrolidin-1-yl-methanone,
(2,3-dihydro-indol-1-yl)-{(5S*)-5-[4-(2-isopropoxy-phenyl)-piperazin-1-ylmethyl]-4,5-dihydro-isoxazol-3-yl}-methanone,
(2,3-dihydro-indol-1-yl)-{(5R*)-5-[4-(2-isopropoxy-phenyl)-piperazin-1-ylmethyl]-4,5-dihydro-isoxazol-3-yl}-methanone,
{(5R*)-5-[4-(2-isopropoxy-phenyl)-piperazin-1-ylmethyl]-4,5-dihydro-isoxazol-3-yl}-(4-phenoxy-piperidin-1-yl)-methanone,
{(5S*)-5-[4-(2-isopropoxy-phenyl)-piperazin-1-ylmethyl]-4,5-dihydro-isoxazol-3-yl}-(4-phenoxy-piperidin-1-yl)-methanone,
[4-(4-fluoro-benzoyl)-piperidin-1-yl]-{(5S*)-5-[4-(2-isopropoxy-phenyl)-piperazin-1-ylmethyl]-4,5-dihydro-isoxazol-3-yl}-methanone,
[4-(4-fluoro-benzoyl)-piperidin-1-yl]-{(5R*)-5-[4-(2-isopropoxy-phenyl)-piperazin-1-ylmethyl]-4,5-dihydro-isoxazol-3-yl}-methanone,
(5-bromo-3,3-dimethyl-2,3-dihydro-indol-1-yl)-{(5R*)-5-[4-(2-isopropoxy-phenyl)-piperazin-1-ylmethyl]-4,5-dihydro-isoxazol-3-yl}-methanone,
(5-bromo-3,3-dimethyl-2,3-dihydro-indol-1-yl)-{(5S*)-5-[4-(2-isopropoxy-phenyl)-piperazin-1-ylmethyl]-4,5-dihydro-isoxazol-3-yl}-methanone,
{(5S*)-5-[4-(2-isopropoxy-phenyl)-piperazin-1-ylmethyl]-4,5-dihydro-isoxazol-3-yl}-morpholin-4-yl-methanone, and
{(5R*)-5-[4-(2-Isopropoxy-phenyl)-piperazin-1-ylmethyl]-4,5-dihydro-isoxazol-3-yl}-morpholin-4-yl-methanone.

10. The compound of claim 9 selected from the group consisting of:
1-{(5R*)-5-[4-(2-isopropoxy-phenyl)-piperazin-1-ylmethyl]-4,5-dihydro-isoxazol-3-ylmethyl}-piperidin-2-one,
1-{(5S*)-5-[4-(2-isopropoxy-phenyl)-piperazin-1-ylmethyl]-4,5-dihydro-isoxazol-3-ylmethyl}-piperidin-2-one,
1-{5-[4-(2-isopropoxy-phenyl)-piperazin-1-ylmethyl]-4,5-dihydro-isoxazol-3-ylmethyl}-2,3-dihydro-1H-indole,
1-{5-[4-(2-isopropoxy-phenyl)-piperazin-1-ylmethyl]-4,5-dihydro-isoxazol-3-ylmethyl}-6-nitro-2,3-dihydro-1H-indole,
1-{5-[4-(2-isopropoxy-phenyl)-piperazin-1-ylmethyl]-4,5-dihydro-isoxazol-3-ylmethyl}-2,3-dihydro-1H-indol-6-ylamine,
(2,6-difluoro-benzyl)-(1-{5-[4-(2-isopropoxy-phenyl)-piperazin-1-ylmethyl]-4,5-dihydro-isoxazol-3-ylmethyl}-2,3-dihydro-1H-indol-6-yl)-amine,
(2-fluoro-benzyl)-(1-{5-[4-(2-isopropoxy-phenyl)-piperazin-1-ylmethyl]-4,5-dihydro-isoxazol-3-ylmethyl}-2,3-dihydro-1H-indol-6-yl)-amine,
(2,3-dihydro-indol-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-(6-nitro-2,3-dihydro-indol-1-yl)-methanone,
{5-[4-(2-isopropoxy-phenyl)-piperazin-1-ylmethyl]-4,5-dihydro-isoxazol-3-yl}-piperidin-1-yl-methanone,
(5-bromo-2,3-dihydro-indol-1-yl)-{5-[4-(2-isopropoxy-phenyl)-piperazin-1-ylmethyl]-4,5-dihydro-isoxazol-3-yl}-methanone,
(6-amino-2,3-dihydro-indol-1-yl)-{5-[4-(2-isopropoxy-phenyl)-piperazin-1-ylmethyl]-4,5-dihydro-isoxazol-3-yl}-methanone,
[6-(2-fluoro-benzylamino)-2,3-dihydro-indol-1-yl]-{5-[4-(2-isopropoxy-phenyl)-piperazin-1-ylmethyl]-4,5-dihydro-isoxazol-3-yl}-methanone,
(5-chloro-2,3-dihydro-indol-1-yl)-{5-[4-(2-isopropoxy-phenyl)-piperazin-1-ylmethyl]-4,5-dihydro-isoxazol-3-yl}-methanone,
[4-(4-fluoro-benzoyl)-piperidin-1-yl]-{5-[4-(2-isopropoxy-phenyl)-piperazin-1-ylmethyl]-4,5-dihydro-isoxazol-3-yl}-methanone,
(5-bromo-3,3-dimethyl-2,3-dihydro-indol-1-yl)-{5-[4-(2-isopropoxy-phenyl)-piperazin-1-ylmethyl]-4,5-dihydro-isoxazol-3-yl}-methanone,
(5-fluoro-2,3-dihydro-indol-1-yl)-{5-[4-(2-isopropoxy-phenyl)-piperazin-1-ylmethyl]-4,5-dihydro-isoxazol-3-yl}-methanone,
(4-benzoyl-piperidin-1-yl)-{5-[4-(2-isopropoxy-phenyl)-piperazin-1-ylmethyl]-4,5-dihydro-isoxazol-3-yl}-methanone,
5-[4-(2-isopropoxy-phenyl)-piperazin-1-ylmethyl]-4,5-dihydro-isoxazole-3-carboxylic acid (1-acetyl-2,3-dihydro-1H-indol-5-yl)-amide,
(5,6-dimethoxy-2,3-dihydro-indol-1-yl)-{5-[4-(2-isopropoxy-phenyl)-piperazin-1-ylmethyl]-4,5-dihydro-isoxazol-3-yl}-methanone,
5-[4-(2-isopropoxy-phenyl)-piperazin-1-ylmethyl]-4,5-dihydro-isoxazole-3-carboxylic acid (1-acetyl-2,3-dihydro-1H-indol-6-yl)-amide,
{5-[4-(2-isopropoxy-phenyl)-piperazin-1-ylmethyl]-4,5-dihydro-isoxazol-3-yl}-(5-methyl-2,3-dihydro-indol-1-yl)-methanone,
(3,4-dihydro-2H-quinolin-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-morpholin-4-yl-methanone,
{5-[4-(2-isopropoxy-phenyl)-piperazin-1-ylmethyl]-4,5-dihydro-isoxazol-3-yl}-(4-phenoxy-piperidin-1-yl)-methanone,
(4-benzenesulfonyl-piperidin-1-yl)-{5-[4-(2-isopropoxy-phenyl)-piperazin-1-ylmethyl]-4,5-dihydro-isoxazol-3-yl}-methanone,
{5-[4-(2-isopropoxy-phenyl)-piperazin-1-ylmethyl]-4,5-dihydro-isoxazol-3-yl}-pyrrolidin-1-yl-methanone,
(2,3-dihydro-indol-1-yl)-{(5R*)-5-[4-(2-isopropoxy-phenyl)-piperazin-1-ylmethyl]-4,5-dihydro-isoxazol-3-yl}-methanone,
{(5S*)-5-[4-(2-isopropoxy-phenyl)-piperazin-1-ylmethyl]-4,5-dihydro-isoxazol-3-yl}-(4-phenoxy-piperidin-1-yl)-methanone,
[4-(4-fluoro-benzoyl)-piperidin-1-yl]-{(5S*)-5-[4-(2-isopropoxy-phenyl)-piperazin-1-ylmethyl]-4,5-dihydro-isoxazol-3-yl}-methanone,
(5-bromo-3,3-dimethyl-2,3-dihydro-indol-1-yl)-{(5R*)-5-[4-(2-isopropoxy-phenyl)-piperazin-1-ylmethyl]-4,5-dihydro-isoxazol-3-yl}-methanone, and
{(5S*)-5-[4-(2-isopropoxy-phenyl)-piperazin-1-ylmethyl]-4,5-dihydro-isoxazol-3-yl}-morpholin-4-yl-methanone.

11. A compound of claim 1 selected from the group consisting of:

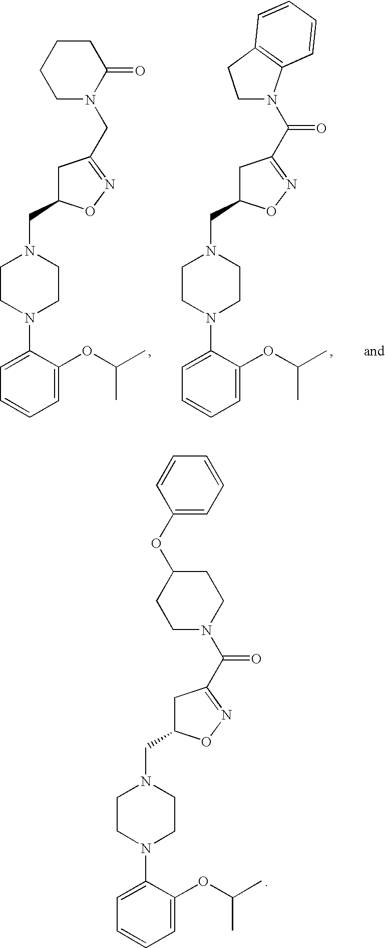

12. The compound of claim 1 and a form thereof in an isolated form.

13. A pharmaceutical composition comprising a pharmaceutically acceptable carrier and an effective amount of a compound of a Formula (I): 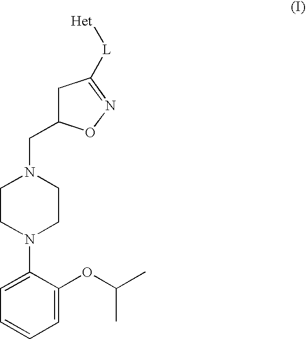
and a form thereof, wherein
L is selected from the group consisting of —CH₂—, —CO— and —CONH—;
Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with C₁₋₈alkyl, amino di-substituted with C₁₋₆alkyl, amino mono-substituted with ArC₁₋₁₆alkyl, amino disubstituted with ArC₁₋₁₆alkyl, Ar, Ar—CO, Ar-oxy, and Ar—SO₂;
Ar is selected from pyridinyl, phenyl and phenyl substituted with one or more substituents selected from halo, C₁₋₆alkyl, and C₁₋₆alkyloxy;
with the proviso that 1-[[4,5-dihydro-5-[[4-[2-(1-methylethoxy)phenyl]-1-piperazinyl]methyl]-3-isoxazolyl]methyl]-2-piperidone is not included.

14. The pharmaceutical composition of claim 13, wherein the effective amount of the compound is in a range of from about 0.001 mg/kg to about 300 mg/kg of body weight per day.

15. A method for treating, ameliorating or preventing an α₁ₐ and/or α_{1d} adrenoreceptor mediated disorder or disease in a subject in need of such treatment, amelioration or prevention comprising administering to the subject a therapeutically or prophylactically effective amount of a compound of a Formula (I): 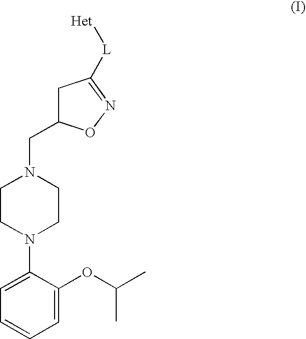
and a form thereof, wherein
L is selected from the group consisting of —CH₂—, —CO— and —CONH—;
Het is heterocyclyl optionally substituted with one, two or three substituents selected from halo, nitro, oxo, C₁₋₆alkyl, C₁₋₆alkyloxy, C₁₋₆alkyl-CO—, amino, amino mono-substituted with C₁₋₈alkyl, amino di-substituted with C₁₋₆alkyl, amino mono-substituted with ArC₁₋₆alkyl, amino disubstituted with ArC₁₋₁₆alkyl, Ar, Ar—CO, Ar-oxy, and Ar—SO₂;
Ar is selected from pyridinyl, phenyl and phenyl substituted with one or more substituents selected from halo, C₁₋₆alkyl, and C₁₋₆alkyloxy;
with the proviso that 1-[[4,5-dihydro-5-[[4-[2-(1-methylethoxy)phenyl]-1-piperazinyl]methyl]-3-isoxazolyl]methyl]-2-piperidone is not included.

16. The method of claim 15, wherein the disorder or disease is Benign Prostatic Hyperplasia.

17. The method of claim 15, wherein the disorder or disease is Lower Urinary Tract Symptoms.

18. The method of claim 15, wherein the compound is 1-{5-[4-(2-isopropoxy-phenyl)-piperazin-1-ylmethyl]-4,5-dihydro-isoxazol-3-ylmethyl}-piperidin-2-one.

19. The method of claim 15, further comprising administering at least the additional therapeutic agent selected from the group consisting of a human testosterone 5-α reductase inhibitor, a 5-α reductase isoenzyme 2 inhibitor, an anti-androgenic agent, an androgen receptor antagonist, a selective androgen receptor modulator, a urinary incontinence drug, an anti-muscarinic agent and a 5HT-receptor modulator.

20. The method of claim 19, wherein at least one additional therapeutic agent is finasteride.
